(19) United States
(12) Patent Application Publication
(10) Pub. No.: US 2005/0119205 A1
(43) Pub. Date: Jun. 2, 2005

(54) Methods of modulating heme oxygenase-1 expression and treating heme oxygenase-1 mediated conditions

(76) Inventor: Mahin D. Maines, Rochester, NY (US)

Correspondence Address: Michael L Goldman;Nixon Peabody, Clinton Square, PO Box 31051, Rochester, NY, 14603, US

(21) Appl. No.: 10/499,243

(22) PCT Filed: Dec. 20, 2002
(86) PCT No.: PCT/US02/41167

Related U.S. Application Data
(60) Provisional application No. 60/342,247, filed on Dec. 21, 2001.

(51) Int. Cl. A61K 48/00; C12N 9/06; C12N 15/85

(52) U.S. Cl. 514/44; 435/455; 435/191

(57) ABSTRACT

A method of modifying heme oxygenase-1 transcription is disclosed. The method includes modifying the nuclear concentration of biliverdin reductase, or fragments or variants thereof which bind to a heme oxygenase-1 regulatory sequence, in a cell, whereby an increase in the nuclear concentration of biliverdin reductase, or fragments or variants thereof, increases the transcription of heme oxygenase-1 and a decrease in the nuclear concentration of biliverdin reductase, or fragments or variants thereof, decreases the transcription of heme oxygenase-1.

This application claims the priority benefit of U.S. Provisional Patent Application Ser. No. 60/342,247 filed Dec. 21, 2001, which is hereby incorporated by reference in its entirety.

The present invention was made, at least in part, with finding received from the National Institutes of Health under grant ES04066. The U.S. government may have certain rights in this invention.

### FIELD OF THE INVENTION

The present invention relates generally to the use of biliverdin reductase, or functional fragments or variants thereof, to modify heme oxygenase-1 (HO-1) expression and thereby treat HO-1 mediated conditions.

### BACKGROUND OF THE INVENTION

The AP-1 site is one of the DNA recognition sequences for leucine zipper proteins. The heme oxygenase cognate, HO-1 or hsp32 (Maines et al., J. Biol. Chem. 261:411-419 (1986)) is activated by increased AP-1 DNA binding in response to certain oxidative stress stimuli (Lee et al., Am. J. Physiol. Lung Mol. Physiol. 279:L175-L182 (2000); He et al., J. Biol. Chem. 276:20858-20865 (2001)). Transcriptional activation involves binding of c-Jun and c-Fos homodimers or heterodimers to the AP-1 site (Angel et al., Biochem. Biophys. Acta 1072:129-157 (1991); Han et al., J. Clin. Invest. 108:73-81 (2001)). Increased AP-1 complex formation is not restricted to HO-1 or oxidative stress, but rather is identified for activation of several oncogenes and kinases in response to cytokines, growth factors, transformation factors, UV radiation and other assortment of stimuli (Devary et al., Mol. Cell Biol. 11:2804-2811 (1991)).

The heme oxygenase system is the most effective mechanism for degradation of heme and generation of biliverdin, carbon monoxide (CO), and iron in the cell. To date, three isozymes, HO-1 (HSP-32), HO-2, and HO-3 have been described. HO-1 and HO-2 are the catalytically active forms and have been well characterized.

Traditionally the HO system was considered only in the context of heme catalytic activity. This view has been revised within the past few years by the finding that the heme metabolites, biliverdin and CO, are biologically active molecules. While it has been suggested that over production of CO and free iron due to uncontrolled upregulation of the HO system may cause cytotoxicity, there is growing evidence of a role for the HO system in cellular defense mechanisms. The bile pigmnent, biliverdin, and its reduction product, bilirubin, have been shown to be potent antioxidants. This is of particular interest since reactive oxygen species are involved in the physiological response to xenobiotics and stress and are a major contributor to an increasing number of disease states, including cancer, cardiovascular disorders, cerebral ischemia, amyotropic lateral sclerosis, Parkinson's and Alzheimer's diseases. Carbon monoxide is a gaseous signaling molecule that, like nitric oxide, is believed to be involved in the regulation of heme containing proteins such as soluble guanylate cyclase.

Other recent studies have identified heme oxygenase-1 derived carbon monoxide is an autocrine inhibitor of vascular smooth muscle cell growth. It has been hypothesized that induction of HO-1 in response to stress may provide protection against oxidative damage. Thus, a controlled upregulation of HO-1 and neurodegenerative disorders might be of particular relevance to the treatment of cardiovascular disease, transplant rejection and Alzheimer's and various neurodegenerative diseases.

Because HO-1 has been implicated in a number of disease states or disorders, it would be desirable to identify the molecular mechanism used for regulatory control over HO-1 production.

The present invention is directed to overcoming these and other deficiencies in the art.

### SUMMARY OF THE INVENTION

A first aspect of the present invention relates to a method of modifying heme oxygenase-1 transcription that includes: modifying the nuclear concentration of biliverdin reductase, or fragments or variants thereof which bind to a heme oxygenase-1 regulatory sequence, in a cell, whereby an increase in the nuclear concentration of biliverdin reductase, or fragments or variants thereof, increases the transcription of heme oxygenase-1 and a decrease in the nuclear concentration of biliverdin reductase, or fragments or variants thereof, decreases the transcription of heme oxygenase-1.

A second aspect of the present invention relates to a method of modifying transcription of a gene including a promoter containing an AP-1 binding region. This method includes: modifying the nuclear concentration of biliverdin reductase, or fragments or variants thereof which bind to an AP-1 binding region, in a cell, whereby an increase in the nuclear concentration of biliverdin reductase, or fragments or variants thereof, increases the transcription of a gene including a promoter which contains an AP-1 binding region and a decrease in the nuclear concentration of biliverdin reductase, or fragments or variants thereof, decreases the transcription of the gene including a promoter which contains the bound AP-1 binding region.

A third aspect of the present invention relates to a method of treating a heme oxygenase-1 mediated condition in a patient that includes: increasing the nuclear concentration of biliverdin reductase, or fragments or variants thereof which bind to a heme oxygenase-1 regulatory sequence, in one or more cells within an affected region of the patient, whereby an increase in the nuclear concentration of biliverdin reductase, or fragments or variants thereof, increases the transcription of heme oxygenase-1, thereby treating the heme oxygenase-1 mediated condition.

A fourth aspect of the present invention relates to a method of treating a heme oxygenase-1 mediated condition in a patient that includes: decreasing the nuclear concentration of biliverdin reductase, or fragments or variants thereof which bind to a heme oxygenase-1 regulatory sequence, in one or more cells within an affected region of the patient, whereby a decrease in the nuclear concentration of biliverdin reductase, or fragments or variants thereof, decreases the transcription of heme oxygenase-1, thereby treating the heme oxygenase-1 mediated condition.

### BRIEF DESCRIPTION OF THE DRAWINGS

FIG. 1 illustrates an amino acid alignment of leucine zipper protein domains. Key leucine zipper domain molecules (L₁-L₅) and their respective replacements are shown in bold. hShaker (SEQ ID NO: 4), hc-Jun (SEQ ID NO: 5), and hc-Fos (SEQ ID NO: 6) have all five (L₁-L₅) leucine molecules, whereas in the case of hBVR (aa 100-157 of SEQ ID NO: 1), rBVR (SEQ ID NO: 7), hc-Myc (SEQ ID NO: 8), sGCN4 (SEQ ID NO: 9), hCREB (SEQ ID NO: 10), and sYAP-1 (SEQ ID NO: 11), leucine molecules at positions L₃, L₃, L₁, L₅, L₅ and L₃ are substituted with lysine, lysine, valine, arginine, lysine and asparagine, respectively. The basic domain is underlined. Sequences are derived from h, Homo sapiens; r, Rattus norvegicus, and s, Saccharomyces cerevisiae (Maines et al., J. Biol. Chem. 261:411-419 (1986); Van Straaten et al., Proc. Natl. Acad. Sci. U.S.A. 80:3183-3187 (1983); Gazin et al., EMBO J. 3:383-387 (1984); Hinnebusch, A. G., Proc. Natl. Acad. Sci. U.S.A. 81:6442-6446 (1984); Bohmann et al., Science 238:1386-1392 (1987); Hoeffler et al., Science 242:1430-1433 (1988); Moye-Rowley et al., Genes Dev. 3: 283-292 (1989); Grupe et al., EMBO J. 9:1749-1756 (1990), each of which is hereby incorporated by reference in its entirety).

FIG. 2 is a predicted three dimensional structure of hBVR. Rat BVR coordinates were used to model three dimensional structure of hBVR. The residues at positions L129, L136, K143, L150, and L157 are indicated and the domain is shown in space filled model. Residues between L129-K143 are predicted to form an α-helix; those between K143-L157 form a β-sheet. N and C denote the N- and C-terminals, respectively. The figure was generated with the molecular graphic program RasMol (Ahmad et al., J. Biol. Chem. 276:18450-18456 (2001), which is hereby incorporated by reference in its entirety).

FIGS. 3 illustrates the detection of high molecular weight protein synthesized by hBVR mRNA. In vitro translated hBVR was visualized on a 12% native-polyacrylamide gel. From left, the first two lanes contained translated hBVR. The molecular weight of the translated protein was approximated to be 69 kDa. This value was obtained using high molecular weight native markers. The third lane is that of the control, which consisted of rabbit reticulocyte lysate with all components present in the translation system minus hBVR mRNA.

FIGS. 4A-B illustrate the identification of the In vitro translated proteins as hBVR by Western blot analysis. FIG. 4A shows an SDS-polyacrylamide gel electrophoresis of in vitro translated BVR with two different amounts of lysate loaded. A 12% SDS gel was used for this experiment. The loading was not intended to be quantitative. Standard molecular weight protein markers indicated the apparent molecular weight of the translated protein bands being 39.9 and 34.6 kDa. FIG. 4B shows a Western blot analysis of in vitro translated hBVR. The first lane contained the translated HBVR, the second contained the wild type E. coli expressed and purified hBVR. The primary antibody was rabbit anti-human kidney BVR. The difference in size of the images shown in FIGS. 4A-B is due to the differential treatment of gels that were used for visualization of translated protein. T denotes in vitro translated while Wt is for wild type hBVR.

FIGS. 5A-C illustrate the results of an HBVR DNA binding assay. Binding assay was carried out using in vitro translated hBVR or HO-1 with modifications denoted for each lane. In FIG. 5A, the first two lanes from the left are controls containing the rabbit lysate but without hBVR mRNA. hBVR binding to the 56-mer DNA with one AP-1 site and binding to 100-mer DNA fragment with two AP-1 sites are shown in the 3^{rd} and 4^{th} lanes, respectively. The 56-mer DNA used in this experiment has been shown to bind with c-Jun/c-Fos heterodimer (Shibahara et al., J. Biol. Chem. 262:12889-12892 (1987), which is hereby incorporated by reference in its entirety). The sequence of the 100-mer long DNA fragment is that of the mouse HO-1 promoter region. FIG. 5B shows an analysis of hBVR binding to the 100-mer DNA fragment with one or zero AP-1 sites. FIG. 5C shows translated HO-1 binding (THO-1) to the 56-mer and 100-mer DNA fragments with one or two AP-1 sites, respectively. Also, binding of purified HO-1 to 100-mer DNA with two or zero AP-1 are shown. For comparison binding of BVR to 100-mer DNA with two or zero AP-1 are shown.

FIG. 6 shows that several mutant hBVR proteins do not form a DNA complex. Binding of the three in vitro translated hBVR mutants denoted in the figure and umnutated control to 100-mer DNA having two AP-1 or zero AP-1 sites are shown. For comparison, binding of native in vitro translated hBVR with DNA having two AP-1 sites and with zero AP-1 sites are also shown.

FIGS. 7A-B illustrate the results of a Northern blot analysis of HO-1 response to inducers in COS cells transfected with antisense hBVR. COS cells were stably transfected with HBVR antisense mRNA as described in the Examples and then used for BVR activity analysis and response of HO-1 to inducers. FIG. 7A shows BVR activity measured in COS cell cytosol fraction prepared from cells pooled from three plates. Enzyme activity was measured as described in the Examples. FIG. 7B shows the Northern blot analysis carried out as described in the Examples using three plates; whole cell preparations were used for isolation of polyA+ RNA. The concentration of MD was 100 μM, while the concentration of heme was 10 μM. The duration of treatment for MD was 30 min followed by a 3 h recovery period. The duration of treatment with heme was 3 h (Keyse et al., Mol Cell Biol. 10:4967-4969 (1990), which is hereby incorporated by reference in its entirety). The control HO-1 signal intensity is arbitrarily designated. Relative intensities, expressed as—fold increase, when compared with the control are 1: antisense+heme=34.4; control+heme=35.4; antisense+MD=7.4 and control+MD=20.

FIG. 8 illustrates the effects of a pair of mutations on the DNA binding capability of human BVR. The results show that the combined glycine¹⁷ plus serine¹⁴⁹ mutations to alanine suppresses binding of biliverdin reductase to DNA.

FIG. 9 illustrates the effects of several mutations on the DNA binding capability of human BVR. The results show that serine¹⁴⁹ in the kinase domain, but not glycine¹⁷ in the nucleotide binding motif, is essential to biliverdin reductase—DNA complex formation.

### DETAILED DESCRIPTION OF THE INVENTION

The present invention relates to the use of biliverdin reductase (“BVR”) or the absence thereof to regulate expression of heme oxygenase-1 (“HO-1”) and other proteins whose expression depends on the DNA-binding of BVR to AP-1 sites within the upstream promoter regions. As a consequence, by modifying the nuclear concentration of BVR, or fragments or variants thereof, the expression of HO-1 and other proteins under regulatory control of AP-1 sites can be regulated, i.e., either enhanced or suppressed.

To increase the nuclear concentration of BVR, or fragments or variants thereof, either BVR or the fragments or variants thereof can be introduced into the cell directly or expressed therein via in vivo cell transformation. To decrease the nuclear concentration of BVR, antisense BVR RNA is introduced into the cell directly or expressed therein via in vivo transformation, which antisense BVR RNA inhibits BVR mRNA translation. Thus, both protein or RNA delivery systems or gene delivery systems can be employed in the present invention.

As used herein, the terms biliverdin reductase and BVR refer to any mammalian BVR, but preferably human BVR (“hBVR”).

One form of HBVR has an amino acid sequence corresponding to SEQ ID NO: 1 as follows:

```
Met Asn Ala Glu Pro Gln Arg Lys Phe Gly Val Val Val Val Gly Val
    1 5 10 15
Gly Arg Ala Gly Ser Val Arg Met Arg Asp Len Arg Asn Pro His Pro
                          20 25 30
Ser Ser Ala Phe Leu Asn Leu Ile Gly Phe Val Ser Arg Arg Glu Leu
                  35 40 45
Gly Ser Ile Asp Gly Val Gln Gln Ile Ser Leu Glu Asp Ala Leu Ser
          50 55 60
Ser Gln Glu Val Glu Val Ala Tyr Ile Cys Ser Glu Ser Ser Ser His
  65 70 75 80
Glu Asp Tyr Ile Arg Gln Phe Leu Asn Ala Gly Lys His Val Leu Val
                                  85 90 95
Glu Tyr Pro Met Thr Leu Ser Leu Ala Ala Ala Gln Glu Leu Trp Glu
                        100 105 110
Leu Ala Glu Gln Lys Gly Lys Val Leu His Glu Glu His Val Glu Leu
                115 120 125
Leu Met Glu Glu Phe Ala Phe Leu Lys Lys Glu Val Val Gly Lys Asp
        130 135 140
Leu Leu Lys Gly Ser Leu Leu Phe Thr Ser Asp Pro Leu Glu Glu Asp
145 150 155 160
Arg Phe Gly Phe Pro Ala Phe Ser Gly Ile Ser Arg Leu Thr Trp Leu
                                165 170 175
Val Ser Leu Phe Gly Glu Leu Ser Leu Val Ser Ala Thr Leu Glu Glu
                        180 185 190
Arg Lys Glu Asp Gln Tyr Met Lys Met Thr Val Cys Leu Glu Thr Glu
                195 200 205
Lys Lys Ser Pro Leu Ser Trp Ile Glu Glu Lys Gly Pro Gly Leu Lys
        210 215 220
Arg Asn Arg Tyr Leu Ser Phe His Phe Lys Ser Gly Ser Leu Glu Asn
225 230 235 240
Val Pro Asn Val Gly Val Asn Lys Asn Ile Phe Leu Lys Asp Gln Asn
                                245 250 255
Ile Phe Val Gln Lys Leu Leu Gly Gln Phe Ser Glu Lys Glu Leu Ala
                        260 265 270
Ala Glu Lys Lys Arg Ile Leu His Cys Leu Gly Leu Ala Glu Glu Ile
                275 280 285
Gln Lys Tyr Cys Cys Ser Arg Lys
        290 295
```

Heterologous expression and isolation of hBVR is described in Maines et al., Eur. J. Biochem. 235(1-2):372-381 (1996); Maines et al., Arch. Biochem. Biophys. 300(1):320-326 (1993), each of which is hereby incorporated by reference in its entirety. A DNA molecule encoding this form of hBVR has a nucleotide sequence corresponding to SEQ ID NO: 2 as follows:

| | | | | | | |
|---|---|---|---|---|---|---|
| ggggtggcgc | ccggagctgc | acggagagcg | tgcccgtcag | tgaccgaaga | agagaccaag | 60 |
| atgaatgcag | agcccgagag | gaagtttggc | gtggtggtgg | ttggtgttgg | ccgagccggc | 120 |
| tccgtgcgga | tgagggactt | gcggaatcca | cacccttcct | cagcgttcct | gaacctgatt | 180 |
| ggcttcgtgt | cgagaaggga | gctcgggagc | attgatggag | tccagcagat | ttctttggag | 240 |
| gatgctcttt | ccagccaaga | ggtggaggtc | gcctatatct | gcagtgagag | ctccagccat | 300 |
| gaggactaca | tcaggcagtt | ccttaatgct | ggcaagcacg | tccttgtgga | ataccccatg | 360 |
| acactgtcat | tggcggccgc | tcaggaactg | tgggagctgg | ctgagcagaa | aggaaaagtc | 420 |
| ttgcacgagg | agcatgttga | actcttgatg | gaggaattcg | ctttcctgaa | aaaagaagtg | 480 |
| gtggggaaag | acctgctgaa | agggtcgctc | ctcttcacat | ctgacccgtt | ggaagaagac | 540 |
| cggtttggct | tccctgcatt | cagcggcatc | tctcgactga | cctggctggt | ctccctcttt | 600 |
| ggggagcttt | ctcttgtgtc | tgccactttg | gaagagcgaa | aggaagatca | gtatatgaaa | 660 |
| atgacagtgt | gtctggagac | agagaagaaa | agtccactgt | catggattga | agaaaaagga | 720 |
| cctggtctaa | aacgaaacag | atatttaagc | ttccatttca | agtctgggtc | cttggagaat | 780 |
| gtgccaaatg | taggagtgaa | taagaacata | tttctgaaag | atcaaaatat | atttgtccag | 840 |
| aaactcttgg | gccagttctc | tgagaaggaa | ctggctgctg | aaaagaaacg | catcctgcac | 900 |
| tgcctggggc | ttgcagaaga | aatccagaaa | tattgctgtt | caaggaagta | agaggaggag | 960 |
| gtgatgtagc | acttccaaga | tggcaccagc | atttggttct | tctcaagagt | tgaccattat | 1020 |
| ctctattctt | aaaattaaac | atgttgggga | aacaaaaaaa | aaaaaaaaaa | | 1070 |

The open reading frame which encodes hBVR of SEQ ID NO: 1 extends from nt 1 to nt 888.

Another form of hBVR has an amino acid sequence according to SEQ ID NO: 3 as follows:

```
Met Asn Thr Glu Pro Glu Arg Lys Phe Gly Val Val Val Val Gly Val
    1 5 10 15
Gly Arg Ala Gly Ser Val Arg Met Arg Asp Leu Arg Asn Pro His Pro
                          20 25 30
Ser Ser Ala Phe Leu Asn Leu Ile Gly Phe Val Ser Arg Arg Glu Leu
                  35 40 45
Gly Ser Ile Asp Gly Val Gln Gln Ile Ser Leu Glu Asp Ala Leu Ser
          50 55 60
Ser Gln Glu Val Glu Val Ala Tyr Ile Cys Ser Glu Ser Ser Ser His
  65 70 75 80
Glu Asp Tyr Ile Arg Gln Phe Leu Asn Ala Gly Lys His Val Leu Val
                                  85 90 95
Glu Tyr Pro Met Thr Leu Ser Leu Ala Ala Ala Gln Glu Leu Trp Glu
                        100 105 110
Leu Ala Glu Gln Lys Gly Lys Val Leu His Glu Glu His Val Glu Leu
                115 120 125
Leu Met Glu Glu Phe Ala Phe Leu Lys Lys Glu Val Val Gly Lys Asp
        130 135 140
Leu Leu Lys Gly Ser Leu Leu Phe Thr Ala Gly Pro Leu Glu Glu Glu
145 150 155 160
Arg Phe Gly Phe Pro Ala Phe Ser Gly Ile Ser Arg Leu Thr Trp Leu
                                165 170 175
Val Ser Leu Phe Gly Glu Leu Ser Leu Val Ser Ala Thr Leu Glu Glu
                        180 185 190
Arg Lys Glu Asp Gln Tyr Met Lys Met Thr Val Cys Leu Glu Thr Glu
                195 200 205
Lys Lys Ser Pro Leu Ser Trp Ile Glu Glu Lys Gly Pro Gly Leu Lys
        210 215 220
Arg Asn Arg Tyr Leu Ser Phe His Phe Lys Ser Gly Ser Leu Glu Asn
225 230 235 240
Val Pro Asn Val Gly Val Asn Lys Asn Ile Phe Leu Lys Asp Gln Asn
                                245 250 255
Ile Phe Val Gln Lys Leu Leu Gly Gln Phe Ser Glu Lys Glu Leu Ala
                        260 265 270
Ala Glu Lys Lys Arg Ile Leu His Cys Leu Gly Leu Ala Glu Glu Ile
                275 280 285
Gln Lys Tyr Cys Cys Ser Arg Lys
        290 295
```

This hBVR sequence is reported at Komuro et al., NCBI Accession No. G02066, direct submission to the EMBL Data Library (1998), which is hereby incorporated by reference in its entirety. Differences between the hBVR of SEQ ID NO: 1 and the hBVR of SEQ ID NO: 3 are at aa residues 3, 154, 155, and 160. Thus, residue 3 can be either alanine or threorine, residue 154 can be either alanine or serine, residue 155 can be either aspartic acid or glycine, and residue 160 can be either aspartic acid or glutamic acid.

In addition, BVR from other mammals, such as rat (rBVR), have been recombinantly expressed and isolated (Fakhrai et al., J. Biol. Chem. 267(6):4023-4029 (1992), which is hereby incorporated by reference in its entirety). The rBVR of shares about 82% aa identity to the hBVR of SEQ ID NO: 1, with variations in aa residues being highly conserved.

As described in greater detail in co-pending U.S. patent application Ser. No. 09/606,129 to Maines, filed Jun. 28, 2000 (which is hereby incorporated by reference in its entirety), BVR is characterized by an amazingly large number of functional domains and motifs, including without limitation: putative and/or demonstrated phosphorylation sites from aa 15 to 20, aa 21 to 23, aa 44 to 46 or 47, aa 49 to54, aa 58 to 61, aa 64 to 67, aa 78 to 81, aa 79 to 82, aa 189 to 192, aa 207 to 209, aa 214 to 217, aa 222 to 227, aa 236 to 241, aa 245 to 250, aa 267 to 269 or 270, and aa 294 to 296 of SEQ ID NO: 1; a basic N-terminal domain characterized by aa 6 to 8 of SEQ ID NO: 1; a hydrophobic domain characterized by aa 9 to 14 of SEQ ID NO: 1; a nucleotide binding domain characterized by aa 15 to 20 of SEQ ID NO: 1; an oxidoreductase domain characterized by aa 90 to 97 of SEQ ID NO: 1; a leucine zipper spanning aa 129 to 157 of SEQ ID NO: 1; several kinase motifs, including aa 44 to 46, aa 147 to 149, and aa 162 to 164 of SEQ ID NO: 1; a nuclear localization signal spanning aa 222 to 228 of SEQ ID NO: 1; a myristylation site spanning aa 221 to 225 of SEQ ID NO: 1; a zinc finger domain spanning aa 280 to 293 of SEQ ID NO: 1; and several substrate binding domains.

As used herein, BVR variants and fragments can be substituted for BVR either in whole or in part.

Fragments of BVR preferably contain the leucine-zipper motif as listed above and any suitable nuclear localization signal, including the nuclear localization signal described above. Suitable fragments are capable of binding to the AP-1 binding site(s) in the promoter region of genes whose expression are to be modified, such as HO-1. Suitable fragments can be produced by several means.

Subclones of a gene encoding a known BVR can be produced using conventional molecular genetic manipulation for subcloning gene fragments, such as described by Sambrook et al., Molecular Cloning: A Laboratory Manual, Cold Springs Laboratory, Cold Springs Harbor, New York (1989), and Ausubel et al. (ed.), Current Protocols in Molecular Biology. John Wiley & Sons (New York, N.Y.) (1999 and preceding editions), each of which is hereby incorporated by reference in its entirety. The subclones then are expressed in vitro or in vivo in bacterial cells to yield a smaller protein or polypeptide that can be tested for a particular activity, e.g., binding to an AP-1 site as discussed in the Examples.

In another approach, based on knowledge of the primary structure of the protein, fragments of a BVR gene may be synthesized using the PCR technique together with specific sets of primers chosen to represent particular portions of the protein. Erlich et al., Science 252:1643-51 (1991), which is hereby incorporated by reference in its entirety. These can then be cloned into an appropriate vector for expression of a truncated protein or polypeptide from bacterial cells as described above. For example, oligomers of at least about 15 to 20 nt in length can be selected from the nucleic acid molecules of SEQ ID NO: 2 for use as primers.

In addition, chemical synthesis can also be employed using techniques well known in the chemistry of proteins such as solid phase synthesis (Merrifield, J. Am. Chem. Assoc. 85:2149-2154 (1964), which is hereby incorporated by reference in its entirety) or synthesis in homogenous solution (Houbenweyl, Methods of Organic Chemistry ed. E. Wansch, Vol. 15, I and II, Thieme, Stuttgart (1987), which is hereby incorporated by reference in its entirety).

Exemplary fragments include N-terminal, internal, and C-terminal fragments which possess a functional leucine zipper motif alone or in combination with other motifs, such as a nuclear localization signal.

Variants of suitable BVR proteins or polypeptides can also be expressed. Variants may be made by, for example, the deletion, addition, or alteration of amino acids that have either (i) minimal influence on certain properties, secondary structure, and hydropathic nature of the polypeptide or (ii) substantial effect on one or more properties of BVR. Variants of BVR can also be fragments of BVR which include one or more deletion, addition, or alteration of amino acids of the type described above. The BVR variant preferably contains a deletion, addition, or alteration of amino acids within one of the above-listed functional domains. The substituted or additional amino acids can be either L-amino acids, D-amino acids, or modified amino acids, preferably L-amino acids. Whether a substitution, addition, or deletion results in modification of BVR variant activity may depend, at least in part on whether the altered amino acid is conserved. Conserved amino acids can be grouped either by molecular weight or charge and/or polarity of R groups, acidity, basicity, and presence of phenyl groups, as is known in the art.

Exemplary variants include the protein or polypeptides of SEQ. ID. Nos. 1 and 3, which have single or multiple amino acid residue substitutions, including, without limitation, SEQ ID NO: 1 as modified by one or more of the following variations: (i) Gly¹⁷→Ala within the nucleotide binding domain, (ii) Ser44→Ala within one of the kinase motifs, (iii) Cys⁷⁴→Ala within a substrate binding domain, (iv) Lys⁹²His⁹³→Ala-Ala within the oxidoreductase motif, (v) G²²²LKRNR²²⁷→VIGSTG within the nuclear localization signal, and (vi) Cys²⁸¹→Ala within the zinc finger domain, and Lys²⁹⁶→Ala at the C terminus within a substrate binding domain (i.e., protein kinase inhibitory domain).

Variants may also include, for example, a polypeptide conjugated to a signal (or leader) sequence at the N-terminal end of the protein which co-translationally or post-translationally directs transfer of the protein. The polypeptide may also be conjugated to a linker or other sequence for ease of synthesis, purification, identification, or therapeutic use (i.e., delivery) of the polypeptide.

Another variant type of BVR is a fusion polypeptide that includes a fragment of BVR containing the functional leucine zipper motif (but not the endogenous nuclear localization signal) and a functional nuclear localization signal. The fusion protein can be expressed or synthesized using known techniques in the art. A number of nuclear localization signals have been identified in the art and can be utilized in combination with the fragment of BVR to obtain the fusion protein, which is targeted for uptake into the cell nucleus following its introduction into the cell whose HO-1 levels are to be modified in accordance with the present invention. Production of chimeric genes encoding such fusion proteins can be carried out as described infra.

The BVR protein or polypeptide (or fragment or variant thereof) can be recombinantly produced, isolated, and then purified, if necessary. When recombinantly produced, the biliverdin reductase protein or polypeptide (or fragment or variant thereof) is expressed in a recombinant host cell, typically, although not exclusively, a prokaryote.

When a prokaryotic host cell is selected for subsequent transformation, the promoter region used to construct the recombinant DNA molecule (i.e., transgene) should be appropriate for the particular host. The DNA sequences of eukaryotic promoters, as described infra for expression in eukaryotic host cells, differ from those of prokaryotic promoters. Eukaryotic promoters and accompanying genetic signals may not be recognized in or may not function in a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology. 68:473 (1979), which is hereby incorporated by reference in its entirety

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a Shine-Dalgarno (“SD”) sequence about 7-9 bases 5′ to the initiation codon (“ATG”) to provide a ribosome binding site. Thus, any SD-ATG combination that can be utilized by host cell ribosomes may be employed. Such combinations include, but are not limited to, the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

Mammalian cells can also be used to recombinantly produce BVR or fragments or variants thereof. Mammalian cells suitable for carrying out the present invention include, among others: COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573), CHOP, and NS-1 cells.

Suitable expression vectors for directing expression in mammalian cells generally include a promoter, as well as other transcription and translation control sequences known in the art. Common promoters include SV40, MMTV, metallothionein-1, adenovirus Ela, CMV, immediate early, immunoglobulin heavy chain promoter and enhancer, and RSV-LTR.

Regardless of the selection of host cell, once the DNA molecule coding for a biliverdin reductase protein or polypeptide, or fragment or variant thereof, has been ligated to its appropriate regulatory regions (or chimeric portions) using well known molecular cloning techniques, it can then be introduced into a suitable vector or otherwise introduced directly into a host cell using transformation protocols well known in the art (Sambrook et al., Molecular Cloning: A Laboratory Manual, Second Edition, Cold Spring Harbor Press, NY (1989), which is hereby incorporated by reference in its entirety).

When an expression vector is used for purposes of in vivo transformation to induce or inhibit of BVR expression in a target cell, promoters of varying strength can be employed depending on the degree of enhancement of suppression desired. One of skill in the art can readily select appropriate mammalian promoters based on their strength as a promoter. Alternatively, an inducible promoter can be employed for purposes of controlling when expression or suppression of BVR is desired. One of skill in the art can readily select appropriate inducible mammalian promoters from those known in the art. Finally, tissue specific mammalian promoters can be selected to restrict the efficacy of any gene transformation system to a particular tissue. Tissue specific promoters are known in the art and can be selected based upon the tissue or cell type to be treated.

The recombinant molecule can be introduced into host cells via transformation, particularly transduction, conjugation, mobilization, or electroporation. Suitable host cells include, but are not limited to, bacteria, virus, yeast, mammalian cells, insect, plant, and the like. The host cells, when grown in an appropriate medium, are capable of expressing the biliverdin reductase, or fragment or variant thereof, which can then be isolated therefrom and, if necessary, purified. The biliverdin reductase, or fragment or variant thereof, is preferably produced in purified form (preferably at least about 60%, more preferably 80%, pure) by conventional techniques.

A further aspect of the present invention relates to an antisense nucleic acid molecule capable of hybridizing with an RNA transcript coding for BVR. Basically, the antisense nucleic acid is expressed from a transgene which is prepared by ligation of a DNA molecule, coding for BVR, or a fragment or variant thereof, into an expression vector in reverse orientation with respect to its promoter and 3′ regulatory sequences. Upon transcription of the DNA molecule, the resulting RNA molecule will be complementary to the mRNA transcript coding for the actual protein or polypeptide product. Ligation of DNA molecules in reverse orientation can be performed according to known techniques which are standard in the art.

Such antisense nucleic acid molecules of the invention may be used in gene therapy to treat or prevent various disorders. For a discussion of the regulation of gene expression using anti-sense genes, see Weintraub et al., Reviews-Trends in Genetics, 1(1) (1986), which is hereby incorporated by reference in its entirety. As discussed infra, recombinant molecules including an antisense sequence or oligonucleotide fragment thereof, may be directly introduced into cells of tissues in vivo using delivery vehicles such as retroviral vectors, adenoviral vectors and DNA virus vectors. They may also be introduced into cells in vivo using physical techniques such as microinjection and electroporation or chemical methods such as coprecipitation and incorporation of DNA into liposomes.

By virtue of BVR's role in binding to the AP-1 binding site in the upstream promoter region of various genes, including the AP-1 binding site of HO-1, BVR or fragments or variants thereof as well as antisense BVR RNA, can be used to modify the transcription level of such genes.

Thus, one aspect of the present invention relates to methods of modifying HO-1 transcription (and thus expression levels) or, more generally, modifying the transcription of a gene that includes a promoter containing an AP-1 binding region (preferably two AP-1 binding regions). These are achieved by modifying the nuclear concentration of BVR (or fragments or variants thereof) in a cell, whereby an increase in the nuclear concentration of biliverdin reductase, or fragments or variants thereof, increases the transcription of the gene whose promoter region includes an AP-1 binding site, e.g., heme oxygenase-1; and a decrease in the nuclear concentration of biliverdin reductase, or fragments or variants thereof, decreases the transcription of the gene whose promoter region includes an AP-1 binding site, e.g., heme oxygenase-1.

The cell in which the nuclear concentration of BVR, or fragments or variants thereof, is to be modified can be located in vivo or ex vivo.

The nuclear concentration of BVR (or fragments or variants thereof) can be modified according to a number of approaches, either by delivering the BVR (or fragments or variants thereof) or antisense BVR RNA molecule into the cell in a manner which affords the protein or polypeptide or RNA molecule to be active within the cell or by delivering DNA encoding BVR (or fragments or variants thereof) or antisense BVR RNA molecule into the cell in a manner effective to induce the expression thereof in the cell. When BVR (or fragments or variants thereof) is delivered into target cells, it is desirable that such delivery be effective to cause nuclear uptake of the BVR (or fragments or variants thereof). As noted above, BVR or fragments or variants contain the native BVR nuclear localization signal or a chimeric nuclear localization signal. When antisense BVR RNA is delivered into target cells, the antisense RNA is effective in the cytoplasm and need not be targeted to any particular location within the cytoplasm, although higher efficacy can be obtained when targeting the antisense BVR RNA to ribosomal sites.

One approach for delivering protein or polypeptides or RNA molecules into cells involves the use of liposomes. Basically, this involves providing a liposome which includes that protein or polypeptide or RNA to be delivered, and then contacting the target cell with the liposome under conditions effective for delivery of the protein or polypeptide or RNA into the cell.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989), each of which is hereby incorporated by reference in its entirety). When liposomes are endocytosed by a target cell, for example, they can be routed to acidic endosomes which will destabilize the liposome and result in drug release.

Alternatively, the liposome membrane can be chemically modified such that an enzyme is placed as a coating on the membrane, which enzyme slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release.

This liposome delivery system can also be made to accumulate at a target organ, tissue, or cell via active targeting (e.g., by incorporating an antibody or hormone on the surface of the liposomal vehicle). This can be achieved according to known methods.

Different types of liposomes can be prepared according to Bangham et al., J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., each of which is hereby incorporated by reference in its entirety.

An alternative approach for delivery of proteins or polypeptides involves the conjugation of the desired protein or polypeptide to a polymer that is stabilized to avoid enzymatic degradation of the conjugated protein or polypeptide. Conjugated proteins or polypeptides of this type are described in U.S. Pat. No. 5,681,811 to Ekwuribe, which is hereby incorporated by reference in its entirety.

Yet another approach for delivery of proteins or polypeptides involves preparation of chimeric proteins according to U.S. Pat. No. 5,817,789 to Heartlein et al., which is hereby incorporated by reference in its entirety. The chimeric protein can include a ligand domain and, e.g., BVR or a fragment or variant thereof as described above. The ligand domain is specific for receptors located on a target cell. Thus, when the chimeric protein is delivered intravenously or otherwise introduced into blood or lymph, the chimeric protein will adsorb to the targeted cell, and the targeted cell will internalize the chimeric protein.

When it is desirable to achieve heterologous expression of a desirable protein or polypeptide or RNA molecule in a target cell, DNA molecules encoding the desired protein or polypeptide or RNA can be delivered into the cell. Basically, this includes providing a nucleic acid molecule encoding the protein or polypeptide and then introducing the nucleic acid molecule into the cell under conditions effective to express the protein or polypeptide or RNA in the cell. Preferably, this is achieved by inserting the nucleic acid molecule into an expression vector before it is introduced into the cell.

When transforming mammalian cells for heterologous expression of a protein or polypeptide, an adenovirus vector can be employed. Adenovirus gene delivery vehicles can be readily prepared and utilized given the disclosure provided in Berkner, Biotechniques 6:616-627 (1988) and Rosenfeld et al., Science 252:431-434 (1991), WO 93/07283, WO 93/06223, and WO 93/07282, each of which is hereby incorporated by reference in its entirety. Adeno-associated viral gene delivery vehicles can be constructed and used to deliver a gene to cells. The use of adeno-associated viral gene delivery vehicles in vitro is described in Chatterjee et al., Science 258:1485-1488 (1992); Walsh et al., Proc. Nat'l. Acad. Sci. 89:7257-7261 (1992); Walsh et al., J. Clin Invest. 94:1440-1448 (1994); Flotte et al., J. Biol. Chem. 268:3781-3790 (1993); Ponnazhagan et al., J. Exp. Med. 179:733-738 (1994); Miller et al., Proc. Nat'l Acad. Sci. 91:10183-10187 (1994); Einerhand et al., Gene Ther. 2:336-343 (1995); Luo et al., Exp. Hematol. 23:1261-1267 (1995); and Zhou et al., Gene Ther. 3:223-229 (1996), each of which is hereby incorporated by reference in its entirety. In vivo use of these vehicles is described in Flotte et al., Proc. Nat'l Acad. Sci. 90:10613-10617 (1993); and Kaplitt et al., Nature Genet. 8:148-153 (1994), each of which is hereby incorporated by reference in its entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; and U.S. Pat. No. 5,885,808 to Spooner et al.; and U.S. Pat. No. 5,871,727 to Curiel, each of which is hereby incorporated by reference in its entirety).

Retroviral vectors which have been modified to form infective transformation systems can also be used to deliver nucleic acid encoding a desired protein or polypeptide or RNA product into a target cell. One such type of retroviral vector is disclosed in U.S. Pat. No. 5,849,586 to Kriegler et al., which is hereby incorporated by reference in its entirety.

Regardless of the type of infective transformation system employed, it should be targeted for delivery of the nucleic acid to a specific cell type. For example, for delivery of the nucleic acid into a cluster of cells, a high titer of the infective transformation system can be injected directly within the site of those cells so as to enhance the likelihood of cell infection. The infected cells will then express the desired product, in this case BVR (or fragments or variants thereof) or antisense BVR RNA, to modify the expression of genes containing AP-1 binding sites in their promoter region such as HO-1.

Whether the proteins or polypeptides or nucleic acids are administered alone or in combination with pharmaceutically or physiologically acceptable carriers, excipients, or stabilizers, or in solid or liquid form such as, tablets, capsules, powders, solutions, suspensions, or emulsions, they can be administered orally, parenterally, subcutaneously, intravenously, intramuscularly, intraperitoneally, by intranasal instillation, by intracavitary or intravesical instillation, intraocularly, intraarterially, intralesionally, by application to mucous membranes, such as, that of the nose, throat, and bronchial tubes, or by transdermal delivery. For most therapeutic purposes, the proteins or polypeptides or nucleic acids can be administered intravenously.

For injectable dosages, solutions or suspensions of these materials can be prepared in a physiologically acceptable diluent with a pharmaceutical carrier. Such carriers include sterile liquids, such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

For use as aerosols, the proteins or polypeptides or nucleic acids in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

Both the biliverdin reductase, or fragment or variant thereof, and the antisense RNA can be delivered to the target cells (i.e., at or around the site of the stroke/ischemic event) using the above-described methods for delivering such therapeutic products. In delivering the therapeutic products to nerve cells in the brain, consideration should be provided to negotiation of the blood-brain barrier. The blood-brain barrier typically prevents many compounds in the blood stream from entering the tissues and fluids of the brain. Nature provides this mechanism to insure a toxin-free environment for neurologic function. However, it also prevents delivery to the brain of therapeutic compounds.

One approach for negotiating the blood-brain barrier is described in U.S. Pat. No. 5,752,515 to Jolesz et al., which is hereby incorporated by reference in its entirety. Basically, the blood-brain barrier is temporarily “opened” by targeting a selected location in the brain and applying ultrasound to induce, in the central nervous system (CNS) tissues and/or fluids at that location, a change detectable by imaging. A protein or polypeptide or RNA molecule of the present invention can delivered to the targeted region of the brain while the blood-brain barrier remains “open,” allowing targeted neuronal cells to uptake the delivered protein or polypeptide or RNA. At least a portion of the brain in the vicinity of the selected location can be imaged, e.g., via magnetic resonance imaging, to confirm the location of the change. Alternative approaches for negotiating the blood-brain barrier include chimeric peptides and modified liposome structures which contain a PEG moiety (reviewed in Pardridge, J. Neurochem. 70:1781-1792 (1998), which is hereby incorporated by reference in its entirety), as well as osmotic opening (i.e., with bradykinin, mannitol, RPM7, etc.) and direct intracerebral infusion (Kroll et al., Neurosurgery 42(5):1083-1100 (1998), which is hereby incorporated by reference in its entirety.

HO-1 has been implicated in a number of dysfunctional states or conditions in a variety of tissues and cell types and regulation of HO-1 levels in targeted cells or tissues has been reported or described to provide therapeutic effect for such dysfunctional states or conditions.

HO-1 underexpression has been implicated in the following dysfunctional states or conditions: chronic inflammatory diseases; hypoxia-associated ocular complications (see Deramaudt et al., J. Cell Biochem. 68(1): 121-127 (1998), which is hereby incorporated by reference in its entirety) including corneal inflammation, ulcerative keratitis, infection, neovascularization, epithelial microcysts, and endothelial polymegathism; fetal growth problems (see Kreiser et al. Laboratory Investigation 82:687-92 (2002), which is hereby incorporated by reference in its entirety); hyperoxia in pulmonary epithelial cells (Lee et al., Proc. Natl. Acad. Sci. USA 93:10393-10398 (1996); xenograft or allograft survival following transplantation (see Niimi et al., Surgery 128:910-917 (2000), Brouard et al., Transplantation 67:1614-1618 (1999), Soares et al., Nature Medicine 4:1073-1077 (1998), and Woo et al., Transplant Immunology 6:84-93 (1998), each of which is hereby incorporated by reference in its entirety); acute heme protein-induced toxicity (Nath et al., Am. J. Pathol. 156:1527-1535 (2000), which is hereby incorporated by reference in its entirety); high vascular resistance disorders including hypertension, primary or secondary vasospasm, angina pectoris, ischemia and reperfusion injury, and preeclampsia of pregnancy (see Ono et al., J. Neurosurgery 96:1094-1102 (2002), Shen et al, Transplantation 74:315-319 (2002), Katori et al., Transplantation 73:287-292 (2002), Inguaggiato et al., Kidney International 60:2181-2191 (2001), Salahudeen et al., Transplantation 72:1498-1504 (2001), Panahian et al., J. Neurochemistry 72:1187-1203 (1999), and U.S. Pat. No. 5,217,977 to Levere et al., each of which is hereby incorporated by reference in its entirety); bronchial asthma (see U.S. Pat. No. 5,217,977 to Levere et al., which is hereby incorporated by reference in its entirety); inflammation (see Wagener et al., Blood 98:1802-1811 (2001), and U.S. Pat. No. 6,066,333 to Willis et al., each of which is hereby incorporated by reference in its entirety); restenosis or other conditions involving vascular smooth muscle cell proliferation (see U.S. Pat. No. 6,203,991 to Nabel et al., which is hereby incorporated by reference in its entirety); conditions associated with non-necrotizing thermal injury such as sunburn and other conditions caused by excessive exposure to ultraviolet irradiation; actinic keratitis; primary burns caused by, e.g., exposure to hot or boiling water or hot metal surfaces (see U.S. Pat. Nos. 5,674,505 and 5,783,201 both to Levere et al., which are hereby incorporated by reference in their entirety); conditions characterized by skin eruptions and/or inflammation, including herpes simplex and herpes zoster, psoriasis, acne, etc. (see U.S. Pat. Nos. 5,674,505 and 5,783,201 both to Levere et al., which are hereby incorporated by reference in their entirety); chapped skin and lips, athlete's foot, minor abrasions linked to inflammation, abrasions from minor skin injuries such as shaving abrasions, etc. (see U.S. Pat. Nos. 5,674,505 and 5,783,201 both to Levere et al., which are hereby incorporated by reference in their entirety); and ulcerations of the mucus membranes, such as oral, nasal and rectal mucosa, oral conditions such as gingivitis, etc. (see U.S. Pat. Nos. 5,674,505 and 5,783,201 both to Levere et al., which are hereby incorporated by reference in their entirety).

Thus, a further aspect of the present invention relates to a method of treating a heme oxygenase-1 mediated condition in a patient by increasing the nuclear concentration of biliverdin reductase, or fragments or variants thereof, in one or more cells within an affected region of the patient, whereby an increase in the nuclear concentration of biliverdin reductase, or fragments or variants thereof, increases the transcription of heme oxygenase-1, thereby treating the heme oxygenase-1 mediated condition.

HO-1 overexpression has been implicated in the following dysfunctional states or conditions: immunosuppressive conditions (see U.S. Pat. No. 6,066,333 to Willis et al., which is hereby incorporated by reference in its entirety); sepsis-associated hypotension (see U.S. Pat. No. 5,888,982 to Perrella et al., which is hereby incorporated by reference in its entirety); and hyperbilirubinemia, e.g., in Dubin-Johnson syndrome (see Drummond et al., Pharmacology 56:158-164 (1998), which is hereby incorporated by reference in its entirety).

Thus, a further aspect of the present invention relates to a method of treating a heme oxygenase-1 mediated condition in a patient by decreasing the nuclear concentration of biliverdin reductase, or fragments or variants thereof, in one or more cells within an affected region of the patient, whereby a decrease in the nuclear concentration of biliverdin reductase, or fragments or variants thereof, decreases the transcription of heme oxygenase-1, thereby treating the heme oxygenase-1 mediated condition.

### EXAMPLES

The following examples are provided to illustrate embodiments of the present invention, but they are by no means intended to limit its scope.

Materials & Methods

All the chemicals and biochemicals used herein were of ultra pure quality purchased from Sigma, Aldrich or Gibco BRL chemical companies. Enzymes used in this study: BamHI, BlpI, HindIII, SalI, SmaI, XhoI, T4 DNA ligase, DNA polymerase, and polynucleotide kinase were purchased from New England Biolabs, Gibco BRL or Amersham Pharmacia. [³⁵S] methionine and [³²p] ATP Redivue™ radioisotopes were purchased from Amersham Pharmacia. Redivue™ L-[³⁵S] methionine (Cat # AG 1094) was used for this grade of [35S] methionine because it does not cause the background labeling of the rabbit reticulocyte lysate 42 kDa protein that can occur using other grades of labels (Jackson et al., Methods Enzymol. 96:50-74 (1983), which is hereby incorporated by reference in its entirety).

In vitro Synthesis of Capped RNA Transcript

The full-length BVR fragment was amplified from the plasmid 494 Gex3 (Maines et al., Eur. J. Biochem. 235:372-381 (1996), which is hereby incorporated by reference in its entirety) using oligos OL.507 and OL.508, while HO-1 (Yoshida et al., Eur. J. Biochem. 71:457-464 (1988), which is hereby incorporated by reference in its entirety) was amplified using oligos OL.547 and OL.548 (Table 1). They were inserted in the multiple cloning site of pCDNA3 (Invitrogen) between BamHI and XhoI sites. The resultant recombinant DNA were named as p507 and p547. Methods used in the construction of plasmids, including restriction enzyme digestion, separation of plasmid DNA and restriction fragments on agarose gels, ligation of DNA fragments, and the isolation of plasmid DNA are described in Sambrook et al. (Sambrook et al., Molecular Cloning: A Laboratory Manual Cold Spring Harbor Laboratory, Cold Spring Harbor, N.Y. (1989), which is hereby incorporated by reference in its entirety). E. coli transformations were performed with CaCl₂ (Cohen et al., Proc. Natl. Acad. Sci. USA. 69:2110-2114 (1972), which is hereby incorporated by reference in its entirety). Polymerase chain reaction (PCR) was carried out as described by Saiki et al. (Saiki et al., Science 239:487-491 (1988), which is hereby incorporated by reference in its entirety). Both Plasmid p507 and p547 were transformed in INV competent cells. The plasmid purification was done with Qiagen Mini Prep plasmid purification kit and was linearized by digesting with SmaI. Linearized plasmid was then PCI (phenol:chloroform:isoamylalcohol; 25:24: 1) treated and ethanol precipitated. Plasmids were dissolved and stored in RNase free water. RNA was transcribed by using RiboProbe in vitro Transcription System from Promega. 5μg linearized template DNA was used in 50 μl reaction volume using T7 RNA polymerase in the presence of m7G cap analog so as to generate the capped transcript. 50-units of ribonuclease inhibitor were also added to the reaction along with appropriate amounts of dithiothreitol (DTT) and nucleotides. After 1 h incubation at 37° C., the reaction mixture was treated with RNase-free DNase (1 μl/μg of template DNA) and extracted with PCI, precipitated with ethanol and ammonium acetate, resuspended in 20 μl RNase free water, and stored at −70° C.

In vitro Translation

A 5.4 kb pcDNA 3 with 1 kb coding HBVR was used as vector to generate in vitro transcribed mRNA with T7 RNA polymerase. The transcribed mRNA was translated in the presence of [35S] methionine using rabbit reticulocyte lysate. In vitro translation was performed using micrococcal nuclease treated rabbit reticulocyte lysate (Promega). 50 μl reaction mixture was prepared by using 35 μl lysate, 1 μl of 0.1 M DTT, 2 μl of 1 mM amino acid mixture minus methionine, 1 μl of RNase inhibitor and 5 μl translation grade [³⁵S] methionine. 5 μl transcribed mRNA was added to the above reaction mixture and immediately incubated at 30° C. for 90 minutes. The in vitro translated proteins were resolved on 12% SDS or native polyacrylamide gel along with rainbow or native high molecular weight markers, respectively (Amersham Pharmacia). The gels were fixed in 10% acetic acid and 30% methanol then treated with autoradiography enhancer (Amplify, Amersham Pharmacia) for 30 min and dried under vacuum at 80° C. for 2 h, and autoradiographed at −70° C.

Preparation of [³²P]-Labeled DNA Fragments

A 56 or 100 bp DNA fragment with and without AP-1 sites was used for the DNA binding assay; their sequences are shown in Table 1 (OL.619, OL.620; OL.623-OL.630). Complimentary oligonucleotides were used to generate double stranded DNA fragments. 150 nanogram aliquots of annealed oligonucleotides were radioactively labeled using [γ-³²P] ATP and T4 polynucleotide kinase. The DNA probes were purified with Qiagen Nucleic Acid Purification Kit.

PCR Generated Site Directed Mutagenesis

1 Kb hBVR fragment was cut out from plasmid p507 by SalI. This 1 Kb fragment was used as the template DNA for site directed mutagenesis. Oligos (OL.582-OL.587) used for mutagenesis of hBVR leucine zipper motif at positions K143, L150, and L157 are shown in Table 1. PCR was carried out in two steps. In the first step the substitutions were introduced by using OL.621 or OL.622 in combination with oligos OL.582 and OL.583, OL.584 and OL.585, OL.586 and OL.587 in order to generate K143A, L150A, and L157A, respectively. In the second stage of the reaction, the PCR products from the first stage were used as template DNA and were joined together by using oligos OL.621 and OL.622 (Table 1). Another difference in the two step 30 cycle PCRs was the Tₘ, which was 48° C. in the first reaction and 43° C. in the second. The PCR products, thus formed, were purified with PCR purification kit (Concert) and digested with BlpI and HindIII. The resultant fragments were inserted in p507, which was used as a vector. Ligation was done within the gel by using 1% low melt agarose. The plasmids were amplified in XL-1 Blue cells and isolated by Qiagen mini prep kit. The DNA sequencing of the mutated hBVR segment was carried out with the oligonucleotides OL.582-OL.587 (Table 1) using the ABI PRISM dye Terminator Cycle Sequencing Ready Reaction kit with AmpliTaq DNA polymerase (Big Dye).

Native and Denaturing Gel Analyses

In vitro translated protein was assayed on native gel immediately after synthesis. One μl of in vitro translated material was added to 2 μl (25 ng) of annealed, unlabelled control DNA fragment. To this 0.4 μg of poly dI•dC (Amersham Pharmacia) in 14 μl DNA binding buffer (10 mM Tris-chloride [pH 7.4], 50 mM NaCl, 1 mM MgCl₂, 1 mM EDTA, 1 mM DTT, 5% glycerol) was added. It was incubated for 5 min at room temperature and after adding 5 μl loading buffer (1.5×DNA binding buffer with bromophenol blue dye) samples were resolved on 12% native polyacrylamide gel in TAE buffer at 35 milliamps. The control DNA helps to prevent the formation of nonspecific protein aggregates, thereby increasing the resolution of protein bands (Halazonetis et al., Cell 55:917-924 (1988), which is hereby incorporated by reference in its entirety). A portion of the translated protein was treated with SDS and analyzed on denaturing 12% polyacrylamide gel.

DNA Binding Assay

As with native gel analysis, in vitro translated proteins were assayed for DNA binding immediately after synthesis. 1 μl of translated material was added to 5000-500,000 cpm of [³²P]-labeled DNA fragment representing approximately 2-3 ng of DNA. 0.1 μg of poly (dI•dC) in 10 μl of DNA binding buffer was added to the labeled DNA. After incubating samples for 20 min at room temperature, 5 μl loading buffer was added. The samples were resolved on 12% native polyacrylamide gel with 35 m Amp at 4° C. The gels were processed as described above. Dried gels were put on two pieces of film separated by a piece of paper. Autoradiography was done at −70° C. for different time periods.

Western Blot Analysis

For Western blot analysis, the primary antibody was rabbit anti-human kidney BVR (Maines et al., Arch. Biochem. Biophys. 300:320-326 (1993), which is hereby incorporated by reference in its entirety) with ECL detection system RPN 2106 (Amersham Pharmacia). Briefly, in vitro translated hBVR was subjected to 12% SDS polyacrylamide gel, transferred to PVDF transfer membrane (Pall Corporation) and subjected to Western blot analysis as described earlier (Salim et al., J. Biol. Chem. 276:10929-10934 (2001), which is hereby incorporated by reference in its entirety).

COS Cell Transfection and BVR Measurement

A cytotoxicity curve for the drug G418 sulfate (Geneticin), used as a marker for the selection of clonal cell lines, was established for exponentially grown COS cells in DMEM (37° C., 5% CO₂). At a concentration of 440 mg/ml and beyond, the drug was found toxic to the parental cell line. Therefore, the selection medium contained G418 at a concentration of 450 mg/ml. pcDNA3 plasmid containing the antisense sequence was isolated from E. coli cultures using the Bigger Prep DNA Isolation Kit following manufacturer's instructions. Transfection was carried out by electroporation. The following day transfected cells were split 1:2 and seeded on a 100 mm culture dish in the selection medium. The selection process was continued for 8-10 days with the change of selection medium every 2 days. Cells from 3 culture dishes were pooled and were used for BVR enzyme activity measurement and mRNA analysis. BVR activity was measured from increase in absorbance at 450 nm as described before (Kutty et al., J. Biol. Chem. 256:3956-3962 (1981), which is hereby incorporated by reference in its entirety) using bilirubin as the substrate and NADH as the cofactor. The activity is expressed as unit, a unit represents nmol bilirubin formed per minute per milligram of protein.

Northern Blotting

The HO-1 hybridization probe was a 569-base pair HO-1 fragment corresponding to nucleotides 86-654 of rat HO-1 cDNA (Shibahara et al., J. Biol. Chem. 262:12889-12892 (1987), which is hereby incorporated by reference in its entirety). A minimum of three culture dishes were pooled and used for each analysis. Total RNA was extracted from COS cells for preparation of polyA+ RNA that was separated by electrophoresis on denaturing formaldehyde gel, and transferred onto a Nytran membrane. The HO-1 and actin probes were labeled using [α-³²P] dCTP with the Rediprime Random Primer Labeling Kit (Amersham Pharmacia). Prehybridization and hybridization were performed as described previously (Ewing et al., Proc. Natl. Acad. Sci. USA 88:5364-5368 (1991), which is hereby incorporated by reference in its entirety). Blots were probed sequentially with HO-1 and actin. The signals were quantitated using TempDens Platform version 1.0.0 and are expressed relative to that of the control. The control level is arbitrarily given the value of one.

TABLE 1

List of Oligonucleotide Sequences
Used in Binding Studies

SEQ
ID

| | | |
|---|---|---|
| Oligo | NO | Sequence |

| | | |
|---|---|---|
| OL.507 | 12 | GGATCCATGAATGCAGAGCCCGAGAG |
| OL.508 | 13 | CTCGAGAGCTACATCACCTCCTCCTC |
| OL.547 | 14 | GGATCCATGGAGCGCCCACAGCTCG |
| OL.548 | 15 | GCTCGAGTGGCGAAGGATCACCATCGCAGGAGCGGTGT |
| OL.582 | 16 | GAAAAAAGAAGTGGTGGGGGCTGACCTGCTGAAAGGGTCG |
| OL.583 | 17 | CGACCCTTTCAGCAGGTCAGCCCCCACCACTTCTTTTTTC |
| OL.584 | 18 | GACCTGCTGAAAGGGTCGGCCCTCTTCACATCTGACCCG |
| OL.585 | 19 | CGGGTCAGATGTGAAGAGGGCCGACCCTTTCAGCAGGTC |
| OL.586 | 20 | CCTCTTCACATCTGACCGGGCTGAAGAAGACCGGTTTGGCT |
| OL.587 | 21 | AGCCAAACCGGTCTTCTTCAGCCGGGTCAGATGTGAAGAGG |
| OL.619 | 22 | TCCTCAGCTGCTTTTATGCTGTGTCATGGTTGGGAGGGGTG |
| | | ATTAGCAGACAAAGGGAAGACAGATTTTGCGATCCTCCCCT |
| | | CTGTTCCCTCTGCCTCAG |
| OL.620 | 23 | CTGAGGCAGAGGGAACAGAGGGGAGGATCGCAAAATCTGT |
| | | CTTCCCTTTGTCTGCTAATCACCCCTCCCAACCATACACAGC |
| | | ATAAAAGCAGCTGAGGA |
| OL.621 | 24 | CAGCCATGAGGACTACATCAG |
| OL.622 | 25 | AGCCAGTTCCTTCTCAGAGAA |
| OL.623 | 26 | TCCTCAGCTGCTTTTATGCTGTGTCATGGTTGGGAGGGGTG |
| | | ATTAGCAGACAAAGGGAAGACAGATTTTGCTGAGTCACCCT |
| | | CTGTTCCCTCTGCCTCAG |
| OL.624 | 27 | CTGAGGCAGAGGGAACAGAGGGTGACTCAGCAAAATCTGT |
| | | CTTCCCTTTGTCTGCTAATCACCCCTCCCAACCATGACACAG |
| | | CATAAAAGCAGCTGAGGA |
| OL.625 | 28 | TCCTCAGCTGCTTTTATGCGATCCTCTGGTTGGGAGGGGTG |
| | | ATTAGCAGACAAAGGGAAGACAGATTTTGCGATCCTCCCCT |
| | | CTGTTCCCTCTGCCTCAG |
| OL.626 | 29 | CTGAGGCAGAGGGAACAGAGGGGAGGATCGCAAAATCTGT |
| | | CTTCCCTTTGTCTGCTAATCACCCCTCCCAACCAGAGGATCG |
| | | CATAAAAGCAGCTGAGGA |
| OL.627 | 30 | CACTGAGAGAAACTATTACACAAGCCACATTAGCATGACTC |
| | | ATTGTTTCTGATCAG |
| OL.628 | 31 | CTGATCAGAAACAATGAGTCATGCTAATGTGGCTTGTGTAA |
| | | TAGTTTCTCTCAGTG |
| OL.629 | 32 | CACTGAGAGAAACTATTACACAAGCCACATTAGCAGATCCT |
| | | CTTGTTTCTGATCAG |
| OL.630 | 33 | CTGATCAGAAACAAGAGGATCTGCTAATGTGGCTTGTGTAA |
| | | TAGTTTCTCTCAGTG |

The substitutions K143A, L150A, L157A in oligonucleotides OL.582-OL.587 are shown in boldface type and are underlined. The AP-1 sites are also shown in boldface type and are underlined, while the replacements of AP-1 sites by random sequences are shown in boldface type only for the oligonucleotides OL.619, OL.620, and OL.623-OL.630.

### Example 1

### Analysis of Biliverdin Reductase Structure

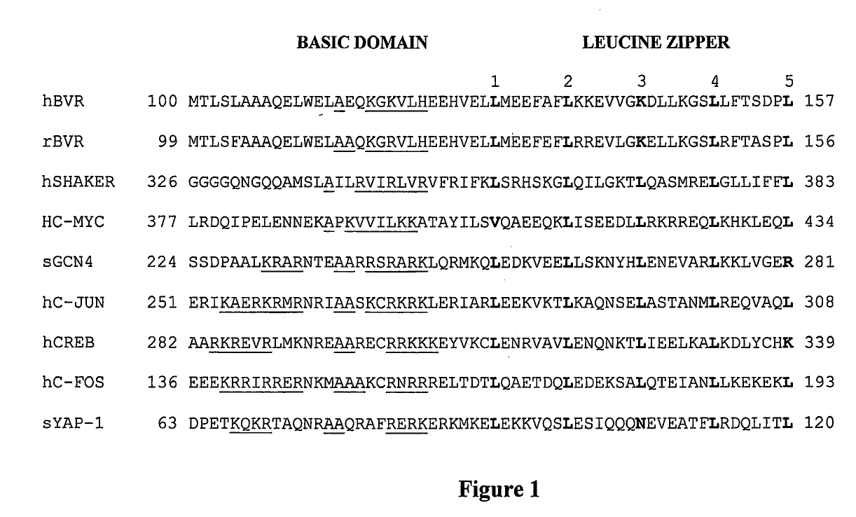

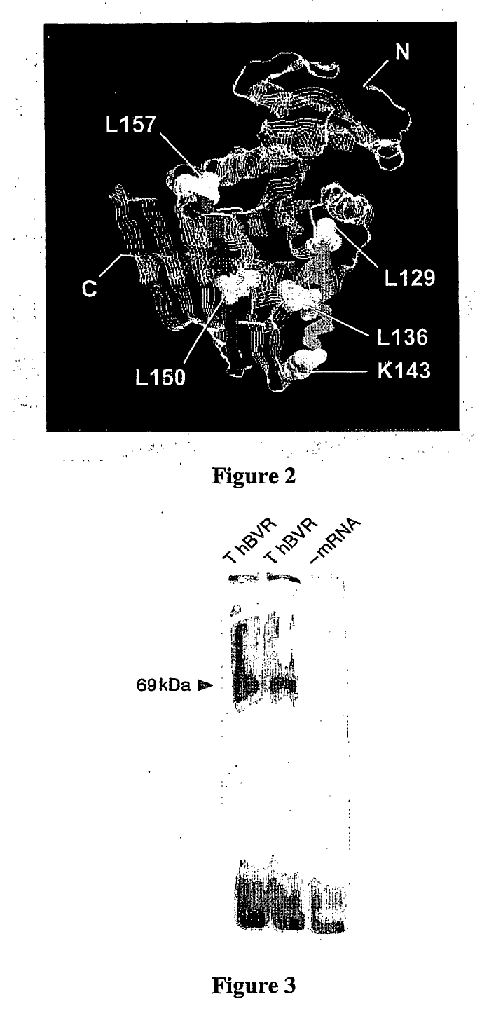

The comparison of the primary structure of hBVR between aa 100-157 to known leucine zipper-type DNA binding proteins shows certain common features (FIG. 1). These include the five repeating amino acids: L₁, L₂, K₃, L₄, L₅ spaced every seventh residue, and a basic domain that is flanked by an upstream alanine residue and starts exactly seven residues N-terminal to L₁. There are however, differences in the primary structure of HBVR and those of most leucine zipper DNA binding proteins: a second basic domain that is present in DNA binding proteins GCN4, c-Jun, c-Fos and YAP-1 is not present in BVR. FIG. 2 shows the secondary structure of HBVR, which is modeled after X-ray diffraction analyses of rBVR crystal structure and shows “U” shaped α-helix-turn-β motif for the leucine zipper motif. Residues that form heptads are identified by space fill model. It is noted that a leucine rich α-helix-turn-β structure is also present in porcine ribonuclease inhibitor and is involved in heterodimer and homodimer formations (Kobe et al., Nature 366:751-756 (1993); Kobe et al., TIBS 19:415-421 (1994), each of which is hereby incorporated by reference in its entirety). On the basis of the crystal structure, Kobe and Deisenhofer have shown that the leucine rich repeat of the ribonuclease inhibitor is also “horseshoe shaped” (Kobe et al., Nature 366:751-756 (1993); Kobe et al., TIBS 19:415-421 (1994), each of which is hereby incorporated by reference in its entirety).

### Example 2

### Human Biliverdin Reductase forms a Homodimer and Binds DNA

Observations with the primary and secondary features of hBVR were followed by examination of whether hBVR forms a dimer, and if so, whether the dimer interacts with DNA. For DNA interaction analysis, a 56-mer and a 100-mer (Table 1) DNA fragments encompassing AP-1 sites were used. The 56-mer fragment was a random fragment with one AP-1 site used for investigation of c-Jun and c-Fos DNA binding (Halazonetis et al., Cell 55:917-924 (1988), which is hereby incorporated by reference in its entirety). AP-1 also has been tested for GCN4 binding (Hope et al., Cell 43:177-188 (1985), which is hereby incorporated by reference in its entirety). The 100-mer DNA fragment corresponded to the HO-1 promoter region encompassing two AP-1 sites (Alam et al., J. Biol. Chem. 269:1001-1009 (1994), which is hereby incorporated by reference in its entirety). In order to bind to DNA, leucine zipper type proteins form a dimer, which takes place at the leucine zipper motif (Busch et al., Trends Genet. 6:36-40 (1990); Johnson et al., Ann. Rev. Biochem. 58:799-839 (1989), each of which is hereby incorporated by reference in its entirety). Most proteins bearing this structural feature form homodimers and dimer formation is typically required for its efficient DNA binding. The only known exception, Fos, forms a stable heterodimer with Jun oncoprotein (Angel et al., Biochem. Biophys. Acta 1072:129-157 (1991), which is hereby incorporated by reference in its entirety). Therefore, hBVR was examined for homodimer formation immediately after in vitro translation of HBVR mRNA, using cold native polyacrylamide gel (4° C.); and employed denaturing/SDS polyacrylamide gel to dissociate the dimer immediately after in vitro translation of HBVR mRNA, should it be formed. On the native gel, the translated protein migrated as an approximate 69 kDa protein (FIG. 3). The protein size was assessed using standard native high molecular weight markers (Amersham Pharmacia). Nonspecific protein aggregation was prevented by addition of control unlabeled DNA (Halazonetis et al., Cell 55:917-924 (1988), which is hereby incorporated by reference in its entirety).

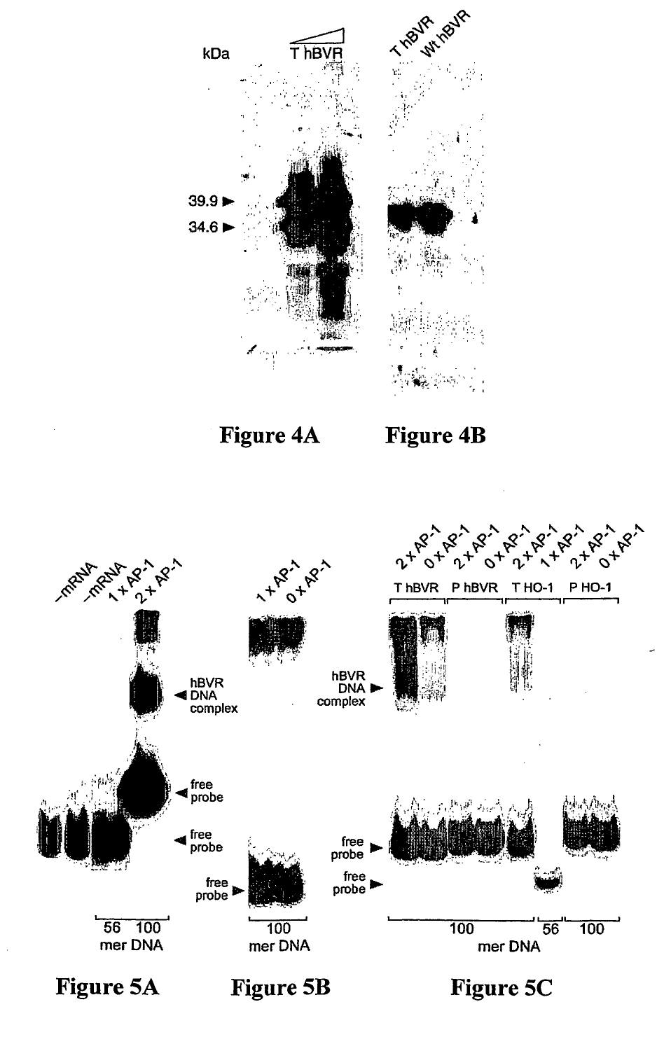

Next, whether the protein synthesized by reticulocyte lysate is in fact hBVR was tested. For this, the in vitro translated protein was examined on a 12% SDS polyacrylamide gel and the gel was processed either for autoradiography (FIG. 4A) or for the Western blot analysis (FIG. 4B). As shown in the autoradiogram, two prominent bands at ˜35 and ˜40 kDa were detected. hBVR, based on its predicted amino acid composition, has a molecular weight of ˜34 kDa (Maines et al., Eur. J. Biochem. 235:372-381 (1996), which is hereby incorporated by reference in its entirety). However, because of extensive posttranslational modification, it migrates as a group of size variants with an approximate molecular weight in the range of ˜38-42 kDa in SDS gel (Maines et al., Eur. J. Biochem. 235:372-381 (1996); Huang et al., J. Biol. Chem. 264:7844-7849 (1989), each of which is hereby incorporated by reference in its entirety). The Western blot shows, when in vitro translated hBVR was probed with antibody to human kidney BVR, two closely migrating bands were observed. The identity of the translated protein was confirmed by comparing its gel migration to wild type hBVR, and immunoreactivity with antibody to purified human kidney BVR. As noted in FIG. 4B, the pattern of immunostaining of proteins was nearly identical. The control consisted of the rabbit reticulocyte lysate without the addition of transcribed hBVR mRNA. In this lysate, bands near the 35-40 kDa region were not detected. Collectively these findings suggested that hBVR is capable of forming a homodimer.

To determine whether the synthesized HBVR binds to DNA, the in vitro translated hBVR was incubated with [³²P]-labeled 56-mer or 100-mer DNA fragments. An identical 56 bp fragment in which the AP-I site was substituted with an unrelated sequence of equal length was used as control DNA. In addition, two identical 100 bp fragments with one AP-1 or zero AP-1 sites were synthesized and used as controls (OL.619, OL.620; OL.623-OL.630, Table 1). After translation, the protein was incubated with DNA fragments and the protein-DNA mixture was run on a native non-denaturing poylacrylamide gel. To differentiate between [³⁵S]-labeled protein and [³²P]-labeled DNA, the processed gel was exposed to two films separated by an opaque piece of paper, with an enhancing screen against the second film. This was to insure that the film next to the gel was exposed to both [³⁵S] and [³²P], while the film next to screen was exposed only to higher energy [³²P] radiation. As shown in FIG. 5A, the translated hBVR did not bind to 56-mer DNA fragment having one AP-1 site, while it did bind to the 100-mer DNA fragment having two AP-1 sites. For these experiments the control contained labeled DNA with rabbit lysate minus hBVR mRNA. As noted in the figure, binding complexes were not detectable in the control lanes. Also, binding of E. coli expressed hBVR protein, which is in monomeric form, to the 100-mer DNA fragment with two AP-1 sites was not detected.

Subsequently, specificity of DNA binding and the number of AP-1 sites used for binding were examined. For this hBVR-AP-1 binding was compared between three 100 bp DNA fragments with two, one, or zero AP-1 sites. As shown in FIG. 5B, hBVR binding occurred when two copies, of the AP-1 binding sequence were present. Interactions of hBVR with 100 bp fragments containing one or zero AP-1 sites were comparable and the subdued signal appeared to reflect AP-1-unrelated DNA-protein interaction. To further examine the specificity of hBVR DNA binding, binding of in vitro translated HO-1 to the same AP-1 containing 56-mer and 100-mer DNA fragments was examined. The larger DNA had two AP-1 sites. Also, DNA binding was examined using E. coli expressed hHO-1 protein. As noted in FIG. 5B, neither the in vitro translated HO-1 nor the purified protein exhibited binding to the DNA fragments. The specificity of binding was assured by addition of control unlabelled 100-mer DNA to all DNA binding experiments that used 100-mer test DNA fragment. The control for the 56-mer test DNA was a 56-mer control unlabeled DNA fragment.

### Example 3

### In vitro Translation of Human Biliverdin Reductase Leucine Zipper Mutants and Their Binding to DNA

To establish the role of the leucine zipper motif of hBVR in DNA-protein interaction, site directed mutagenesis studies were carried out. Mutations were directed to K143, L150 and L157 that were changed to alanine thereby generating K143A, L150A and L157A, respectively. This was a particularly relevant investigation because, as noted above, the model of the secondary structure of hBVR (FIG. 2) predicts a β-sheet structure for hBVR between K143 and L157, while the structure common to most leucine zipper DNA binding proteins is often in entirely α-helical. Studies with Jun and Fos oncoproteins suggest that single mutations in the motif are sufficient to abolish specific DNA binding (Ransone et al., Genes Dev. 3:770-781 (1989), which is hereby incorporated by reference in its entirety). It has also been shown that a single amino acid change in Fos abolishes the DNA binding capabilities of the Fos-Jun dimer complex.

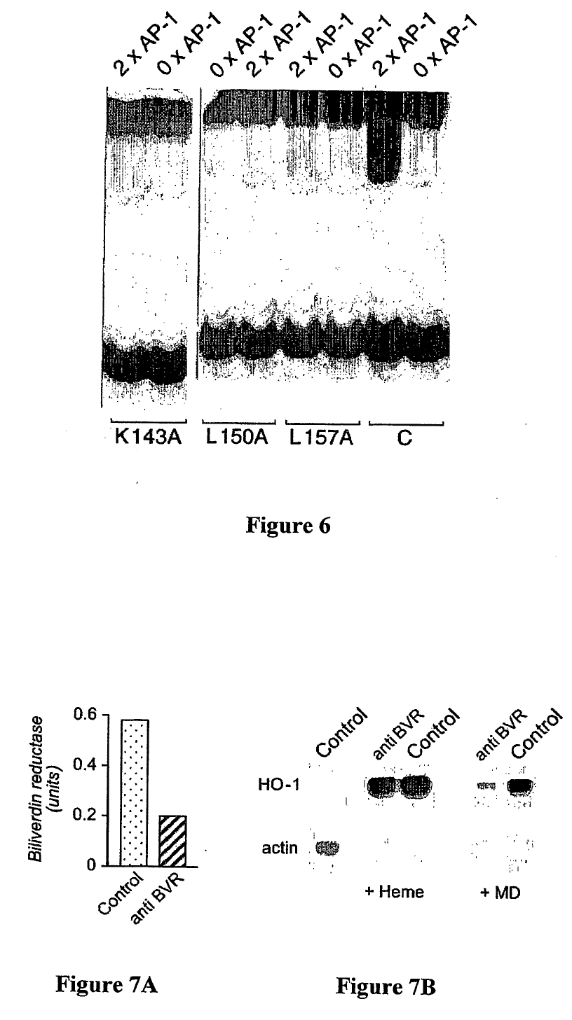

For this set of experiments, the [³⁵S] methionine-labeled mutant BVR proteins were generated by in vitro translation and assayed on a 12% native gel for detection of the ˜69 kDa protein band and analysis of DNA for complex formation. The 100-mer DNA fragment with two AP-1 sites or without an AP-1 site were used. On the native gel the high molecular weight band was not detected with the mutated proteins. Also, as shown in FIG. 6, a single mutation in any of the three positions prevented protein-DNA complex formation. As noted, binding of the three mutant proteins with the DNA fragment having two or zero AP-1 sites was essentially comparable and was similar to that of the native HBVR binding to the 100-bp fragment with no AP-1 site. As before, the control translated hBVR shows clear binding with DNA having two AP-1 sites.

The three-dimensional conformation of hBVR leucine zipper domain, predicted by RasMol molecular graphic program (Ahmad et al., J. Biol. Chem. 276:18450-18456 (2001), which is hereby incorporated by reference in its entirety), suggested that substitution of K143, L150, or L157 by alanine in the leucine zipper motif apparently does not cause conformational changes in the motif and, hence, most likely does not account for the attenuated DNA binding.

### Example 4

### HO-1 Response to Menadione and Heme in COS Cells Transfected with Antisense Human Biliverdin Reductase RNA

To examine whether DNA binding of hBVR has any bearing on gene expression, induction of HO-1 in COS cells stably transfected with antisense hBVR was examined. HO-1 is transcriptionally regulated by a vast array of stimuli that trigger activation of different regulatory factors. Menadione (MD) and heme are both inducers of HO-1 gene expression, but involve distinctly different signaling cascades activating factors. To determine whether the antisense mRNA affected BVR activity, activity in the transfected cells was measured. As shown in FIG. 7A, a 66% decrease in activity was detected. This cell line was then used to examine the response of HO-1 to known inducers, heme and MD, by Northern blot analysis. As noted in FIG. 7B, response of cells carrying antisense HBVR to heme did not differ from that of the control cells and both sets of cells and an increase of approximately 35-fold in HO-1 mRNA was detected. In contrast, MD, which is a generator of oxygen radicals, produced a less than remarkable increase in HO-1 mRNA levels in the transfected cells. The control cells, on the other hand, displayed a robust response to MD. The magnitude of increase in HO-1 mRNA in the control and transfected cells was 20-fold vs. 7-fold, respectively. HO-1 mRNA in COS cells with absence of inducers was marginally detectable.

### Discussion of Examples 1-4

When a leucine zipper motif in the primary sequence of hBVR was detected, in BVR the motif was presumed to be involved either in dimerization, DNA binding, or some other functions related to its kinase activity. Of course, the possibility that the motif is of no apparent biological significance was not ruled out. A unifying feature of sequence specific DNA binding proteins is dimerization. Presently, evidence are provided that indicate formation of a homodimer by HBVR that binds to DNA and involves the leucine repeat region; the DNA binding sites are identified as two AP-1 recognition sequences. The finding that the single form of the nascent protein (FIG. 3) dissociates in two species (FIG. 4A) under denaturing conditions, and identification of the proteins based on their immunoreactivity as BVR (FIG. 4B), are indicative of a BVR homodimer formation. Moreover, the reductase contains the characteristic putative dimerization interface made of L₁, L₂, K₃, L₄, and L₅, which is found in several proteins that bind nucleic acids (FIG. 1). The finding that site directed mutation of these residues blocks the ability of HBVR to form a complex with 100-mer DNA with two AP-1 sites is indicative of their participation in the formation of hBVR DNA complex. Previous studies have shown that in many instances the DNA binding property of proteins with leucine zipper motif is lost with single or double mutations in the motif, which may or may not alter the dimer formation (Halazonetis et al., Cell 55:917-924 (1988); Ransone et al., Genes Dev. 3:770-781 (1989); Hope et al., Cell 46:885-894 (1986), each of which is hereby incorporated by reference in its entirety). In the case of hBVR, individual mutations at the K₃, L₄ and L₅ prevent dimer formation.

Although hBVR has similarities in structure to a number of DNA binding proteins with leucine zipper motif, it also has divergent features. Moreover, based on the predicted secondary structure of HBVR, the sequence of amino acids between L129 to K143 forms a α-helical structure, while the sequence between K143 and L157 is mainly β-sheet. Notably, the predicted secondary structure for many leucine zipper DNA binding proteins is two α-helices separated by a β-turn. The DNA contact region in many of the leucine zipper proteins is the sequence immediately NH₂-terminal to the leucine zipper with a notable degree of basicity that starts seven residues N-terminal to L₁. In BVR, however, the content of basic amino acids in this region is low in comparison with that of other DNA binding proteins, and unlike those proteins that have two clusters of basic residues linked by a spacer sequence with an invariant alanine spacer, only one basic cluster is present in BVR (FIG. 1). A second N-terminal basic domain is also absent from c-Myc, which is a helix-loop-helix DNA binding protein. It has, however, a basic domain near the C-terminus of the protein. The reductase has a basic domain near the carboxyl terminus of the protein: KKRILH (275-280 of SEQ ID NO: 1), which plausibly could also interact with DNA. In addition, the second basic domain is also absent in the leucine zipper protein hShaker K⁺ channel 3 β subunit (FIG. 1); which interestingly is also an oxidoreductase (McCormack et al., Cell 79:1133-1135 (1994), which is hereby incorporated by reference in its entirety). In Shaker, which is a member of the aldo-ketoreductase superfamily, leucine zipper motif is involved in interaction of K⁺ channel subunits and is not believed to have ever been reported to bind to DNA.

Observations with COS transfected with antisense BVR are supportive of the belief that HBVR DNA binding is likely of biological consequence as far as the regulation of HO-1 by free radicals is concerned. The sequence specific DNA binding of BVR to the AP-1 sites of HO-1 is drawn from two pieces of data: 1) BVR-DNA complex formation was observed with a DNA fragment of HO-1 promoter region; and 2) cells transfected with antisense BVR displayed an attenuated increase in HO-1 gene expression in response to oxidative stress, whereas their response to heme was similar to control. As reported, mutations in AP-1 binding sites block HO-1 gene activation by oxidative stimuli (Lee et al., Am. J. Physiol. Lung Mol. Physiol. 279:L175-L182 (2000); He et al., J. Biol. Chem. 276:20858-20865 (2001); Alam et al., J. Biol. Chem. 267:21894-21900 (1992), each of which is hereby incorporated by reference in its entirety). Further, the leucine zipper transcription factors, Jun and Fos, which constitute the AP-1 family, are activated by oxidative events (Devary et al., Cell 71:1081-91 (1992); Minden et al., Cell 81:1147-1157 (1995), each of which is hereby incorporated by reference in its entirety). In addition, several other DNA binding sites for transcriptional activation of HO-1, which is responsive to a wide assortment of stimuli (reviewed in Maines, CRC Press, Inc., Boca Raton, Fla., USA (1992), which is hereby incorporated by reference in its entirety), have been identified (Lee et al., Am. J. Physiol. Lung Mol. Physiol. 279:L175-L182 (2000); He et al., J. Biol. Chem. 276:20858-20865 (2001); Shibahara et al., J. Biol. Chem. 262:12889-12892 (1987); Lavrovsky et al., Proc. Natl. Acad. Sci USA. 91:5987-5991 (1994), each of which is hereby incorporated by reference in its entirety).

Multiple AP-1 binding sites have also been identified in promoter regions of other genes, including without limitation: Monocyte Chemoattractant Protein (MCP-1) (Nakayama et al., J. Immunol. 167(3):1145-1150 (2001), which is hereby incorporated by reference in its entirety); bradykinin B1 receptor (BKB1R) (Yang et al., J. Cellular Biochemistry 82(1):163-170 (2001), which is hereby incorporated by reference in its entirety); Ier5, a member of the slow-kinetics immediate-early gene family (Williams et al., Genomics 55(3):327-334 (1999), which is hereby incorporated by reference in its entirety); and ICR-27, which was obtained from glucocorticoid-resistant human leukemic T cells (Chen et al., J. Biol. Chem. 272(41):25873-25880 (1997), which is hereby incorporated by reference in its entirety).

On the basis of the above-noted observations, it is reasonable to conclude that BVR can function in AP-1 pathway of cell signaling. MD has long been used as an oxidative stress model. It stimulates the rate of NADPH oxidation, H₂O₂ production, and redox cycling that results in formation of superoxide anions (Gillette et al., J. Pharmacol. Expt. Ther. 119:532-540 (1957), which is hereby incorporated by reference in its entirety). The previous findings that the reductase is activated by the oxidant H₂O₂, and is a serine/threonine/tyrosine kinase (Salim et al., J. Biol. Chem. 276:10929-10934 (2001); U.S. patent application Ser. No. 09/606,129 to Maines, filed Jun. 28,2000, each of which is hereby incorporated by reference in its entirety) lend support to this idea. Noteworthy is the fact that H₂0₂ is an activator of HO-1 gene expression Keyse et al., Mol Cell Biol. 10:4967-4969 (1990); Keyse et al., Carcinogenesis 5:787-791 (1990), each of which is hereby incorporated by reference in its entirety). The conclusion that hBVR-DNA binding is linked to the activation of the HO-1 gene is also consistent with previous observations that, in HeLa cells in response to cGMP, and in intact rats in response to LPS, or to the free radical generating compound bromobenzene, reductase translocates from the cytosol to the nucleus (Maines et al., J. Pharmacol. Exp. Ther. 296:1091-1097 (2001), which is hereby incorporated by reference in its entirety). All mentioned stimuli are inducers of HO-1 gene expression.

### Example 5

### Effect of Mutations in Basic Domain and Leucine Zipper Domain on Binding to AP-1 Site of DNA

PCR generated site directed mutagenesis was used to prepare the Gly¹⁷→Ala mutation and the Ser¹⁴⁹→Ala mutations. A 1 Kb HBVR fragment was cut out from plasmid p507 by SalI and used as the template DNA for site directed mutagenesis. Gly¹⁷ was replaced with Ala¹⁷ using primers 732 and 733 shown below:

| | |
|---|---|
| Primer 732 (SEQ ID NO: 34) | |
| GCGGCGGCTG GTGTTGCGCG CGCCGGCTCC GTGCGG | 36 |
| Primer 733 (SEQ ID NO: 35) | |
| CCGCACGGAG CCGGCGCGCG CAACACCAGC CGCCGC | 36 |
| Ser¹⁴⁹ was replaced by alanine residues using | |
| primers 730 and 731 shown below: | |
| Primer 730 (SEQ ID NO: 36) | |
| CCTGCTGAAA GGGGCGGCCG CCGCCGCAGC TGACCCGTTG | 44 |
| GAAG | |
| Primer 731 (SEQ ID NO: 37) | |
| CTTCCAACGG GTCAGCTGCG GCGGCGGCCG CCCCTTTCAG | 44 |
| CAGG | |

The PCR products, thus formed, were purified with PCR purification kit (Concert) and digested with BlpI and HindIII The resultant fragments were inserted in p507, which was used as a vector. Ligation was done within the gel by using 1% low melt agarose. The plasmids were amplified in XL-1 Blue cells and isolated by Qiagen mini prep kit. The DNA sequencing of the mutated HBVR segment was carried out with the oligonucleotides OL.582 and OL.587 (Table 1) using the ABI PRISM dye Terminator Cycle Sequencing Ready Reaction kit with AmpliTaq DNA polymerase (Big Dye).

Preparation of [³²P]-labeled DNA fragments was carried out as described above using OL.623 (Table 1). The DNA binding assay was also carried out as described above.

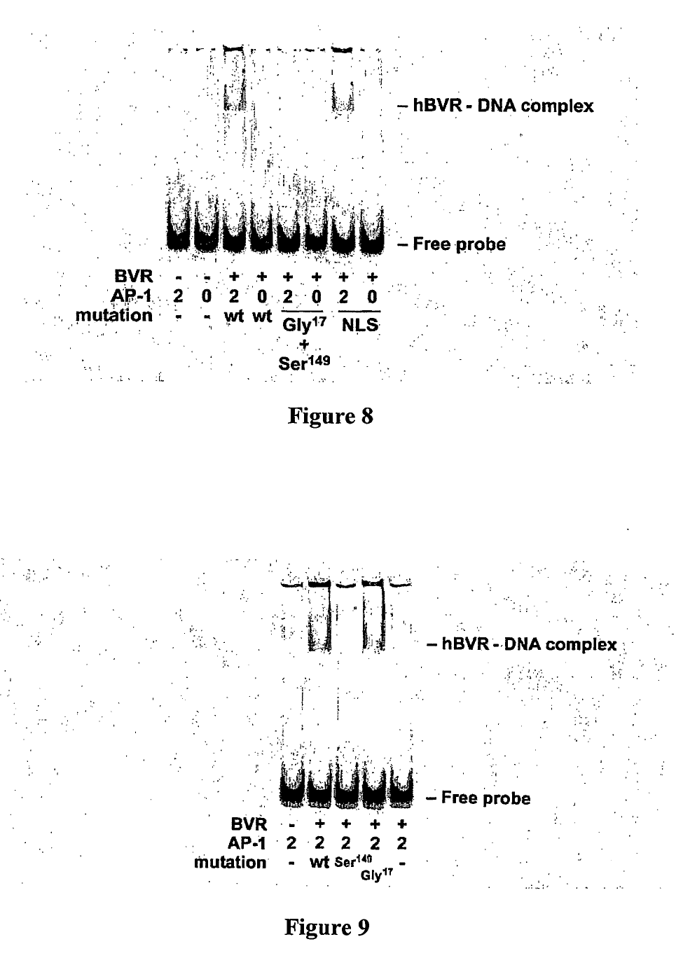

As shown in FIG. 8, a mutant BVR variant containing both the Gly¹⁷→Ala mutation and the Ser¹⁴⁹→Ala mutation was unable to form a BVR-DNA complex. To discern which of the two mutations was responsible for the lack of such binding, single mutants were also tested. As shown in FIG. 9, Ser¹⁴⁹ in the kinase domain (but not Gly¹⁷ in the nucleotide binding motif) is essential for BVR-DNA complex formation.

Although the invention has been described in detail for the purpose of illustration, it is understood that such detail is solely for that purpose, and variations can be made therein by those skilled in the art without departing from the spirit and scope of the invention which is defined by the following claims.

1. A method of modifying heme oxygenase-1 transcription comprising:
modifying the nuclear concentration of biliverdin reductase, or fragments or variants thereof which bind to a heme oxygenase-1 regulatory sequence, in a cell, whereby an increase in the nuclear concentration of biliverdin reductase, or fragments or variants thereof, increases the transcription of heme oxygenase-1 and a decrease in the nuclear concentration of biliverdin reductase, or fragments or variants thereof, decreases the transcription of heme oxygenase-1.

2. The method according to claim 1 wherein said modifying comprises:
transforming the cell with a DNA construct which expresses antisense biliverdin reductase RNA in the cell, said transforming decreasing the nuclear concentration of biliverdin reductase.

3. The method according to claim 1 wherein said modifying comprises:
transforming the cell with a DNA construct which expresses biliverdin reductase or fragments or variants thereof in the cell, said transforming increasing the nuclear concentration of biliverdin reductase or fragments or variants thereof.

4. The method according to claim 1 wherein said modifying comprises:
introducing biliverdin reductase or fragments or variants thereof into the cell under conditions effective to cause nuclear uptake of the biliverdin reductase or fragments or variants thereof.

5. The method according to claim 4 wherein said introducing comprises:
contacting the cell with a delivery vehicle comprising biliverdin reductase or fragments or variants thereof under conditions effective to induce cellular uptake of at least the biliverdin reductase or fragments or variants thereof.

6. The method according to claim 5 wherein the delivery vehicle is a liposome comprising biliverdin reductase or fragments or variants thereof.

7. The method according to claim 5 wherein the delivery vehicle is a fusion protein comprising biliverdin reductase or fragments or variants thereof.

8. The method according to claim 1 wherein the heme oxygenase-1 regulatory sequence is a heme oxygenase-1 promoter region comprising two or more AP-1 binding regions.

9. The method according to claim 1 wherein the cell is ex vivo.

10. The method according to claim 1 wherein the cell is in vivo.

11. A method of modifying transcription of a gene including a promoter containing an AP-1 binding region, said method comprising:
modifying the nuclear concentration of biliverdin reductase, or fragments or variants thereof which bind to an AP-1 binding region, in a cell, whereby an increase in the nuclear concentration of biliverdin reductase, or fragments or variants thereof, increases the transcription of a gene including a promoter which contains an AP-1 binding region and a decrease in the nuclear concentration of biliverdin reductase, or fragments or variants thereof, decreases the transcription of the gene including a promoter which contains the bound AP-1 binding region.

12. The method according to claim 11 wherein said modifying comprises:
transforming the cell with a DNA construct which expresses antisense biliverdin reductase RNA in the cell, said transforming decreasing the nuclear concentration of biliverdin reductase.

13. The method according to claim 11 wherein said modifying comprises:
transforming the cell with a DNA construct which expresses biliverdin reductase or fragments or variants thereof in the cell, said transforming increasing the nuclear concentration of biliverdin reductase or fragments or variants thereof.

14. The method according to claim 11 wherein said modifying comprises:
introducing biliverdin reductase or fragments or variants thereof into the cell under conditions effective to cause nuclear uptake of the biliverdin reductase or fragments or variants thereof.

15. The method according to claim 14 wherein said introducing comprises:
contacting the cell with a delivery vehicle comprising biliverdin reductase or fragments or variants thereof under conditions effective to induce cellular uptake of at least the biliverdin reductase or fragments or variants thereof.

16. The method according to claim 15 wherein the delivery vehicle is a liposome comprising biliverdin reductase or fragments or variants thereof.

17. The method according to claim 15 wherein the delivery vehicle is a fusion protein comprising biliverdin reductase or fragments or variants thereof.

18. The method according to claim 11 wherein the cell is ex vivo.

19. The method according to claim 11 wherein the cell is in vivo.

20. The method according to claim 11 wherein the promoter comprises two or more AP-1 binding regions.

21. The method according to claim 20 wherein the gene is selected from the group of MCP-1, BKB1R, Ier5, ICR-27, and HO-1.

22. A method of treating a heme oxygenase-I mediated condition in a patient comprising:
increasing the nuclear concentration of biliverdin reductase, or fragments or variants thereof which bind to a heme oxygenase-1 regulatory sequence, in one or more cells within an affected region of the patient, whereby an increase in the nuclear concentration of biliverdin reductase, or fragments or variants thereof, increases the transcription of heme oxygenase-1, thereby treating the heme oxygenase-1 mediated condition.

23. The method according to claim 22, wherein the heme oxygenase-1 mediated condition is selected from the group of chronic inflammatory diseases, hypoxia-associated ocular complications, fetal growth problems, hyperoxia in pulmonary epithelial cells, xenograft or allograft survival following transplantation, high vascular resistance disorders, bronchial asthma, inflammation, restenosis or other conditions involving vascular smooth muscle cell proliferation, conditions associated with non-necrotizing thermal injury, conditions characterized by skin eruptions and/or inflammation, chapped skin and lips, athlete's foot, skin abrasions, ulcerations of the mucus membranes, and oral conditions.

24. The method according to claim 22, wherein said increasing comprises:
transforming the one or more cells with a DNA construct which expresses biliverdin reductase or fragments or variants thereof in the one or more cells.

25. The method according to claim 22 wherein said increasing comprises:
introducing biliverdin reductase or fragments or variants thereof into the one or more cells under conditions effective to cause nuclear uptake of the biliverdin reductase or fragments or variants thereof.

26. The method according to claim 25 wherein said introducing comprises:
contacting the one or more cells with a delivery vehicle comprising biliverdin reductase or fragments or variants thereof under conditions effective to induce cellular uptake of at least the biliverdin reductase or fragments or variants thereof.

27. The method according to claim 26 wherein the delivery vehicle is a liposome comprising biliverdin reductase or fragments or variants thereof.

28. The method according to claim 26 wherein the delivery vehicle is a fusion protein comprising biliverdin reductase or fragments or variants thereof.

29. The method according to claim 26 wherein said contacting is carried out by administering the delivery vehicle to the patient under conditions effective to contact the one or more cells.

30. The method according to claim 29 wherein said administering is carried out orally, parenterally, subcutaneously, intravenously, intramuscularly, intraperitoneally, by intranasal instillation, by intracavitary or intravesical instillation, intraocularly, intraarterially, intralesionally, by application to mucous membranes, such as, that of the nose, throat, and bronchial tubes, or by transdermal delivery.

31. A method of treating a heme oxygenase-1 mediated condition in a patient comprising:
decreasing the nuclear concentration of biliverdin reductase, or fragments or variants thereof which bind to a heme oxygenase-1 regulatory sequence, in one or more cells within an affected region of the patient, whereby a decrease in the nuclear concentration of biliverdin reductase, or fragments or variants thereof, decreases the transcription of heme oxygenase-1, thereby treating the heme oxygenase-1 mediated condition.

32. The method according to claim 31, wherein the heme oxygenase-1 mediated condition is selected from the group of immunosuppressive conditions, sepsis-associated hypotension, and hyperbilirubinemia.

33. The method according to claim 31 wherein said decreasing comprises:
transforming the one or more cells with a DNA construct which expresses antisense biliverdin reductase RNA in the one or more cells, said transforming decreasing the nuclear concentration of biliverdin reductase.

34. The method according to claim 31 wherein said decreasing comprises:
introducing antisense biliverdin reductase RNA into the one or more cells.

35. The method according to claim 34 wherein said introducing comprises:
contacting the one or more cells with a delivery vehicle comprising the antisense biliverdin reductase RNA under conditions effective to induce cellular uptake of the antisense biliverdin reductase RNA.

36. The method according to claim 35 wherein the delivery vehicle is a liposome comprising the antisense biliverdin reductase RNA.

37. The method according to claim 35 wherein said contacting is carried out by administering the delivery vehicle to the patient under conditions effective to contact the one or more cells.

38. The method according to claim 37 wherein said administering is carried out orally, parenterally, subcutaneously, intravenously, intramuscularly, intraperitoneally, by intranasal instillation, by intracavitary or intravesical instillation, intraocularly, intraarterially, intralesionally, by application to mucous membranes, such as, that of the nose, throat, and bronchial tubes, or by transdermal delivery.


SEQUENCE LISTING (37 sequences)

SEQ ID NO: 1 - protein, 296 aa, Homo sapiens
MNAEPERKFGVVVVGVGRAGSVRMRDLRNPHPSSAFLNLIGFVSRRELGSIDGVQQISLEDALSSQEVEVAYICSESSSHEDYIRQFLNAGKHVLVEYPMTLSLAAAQELWELAEQKGKVLHEEHVELLMEEFAFLKKEVVGKDLLKGSLLFTSDPLEEDRFGFPAFSGISRLTWLVSLFGELSLVSATLEERKEDQYMKMTVCLETEKKSPLSWIEEKGPGLKRNRYLSFHFKSGSLENVPNVGVNKNIFLKDQNIFVQKLLGQFSEKELAAEKKRILHCLGLAEEIQKYCCSRK

SEQ ID NO: 2 - DNA, 1070 bp, Homo sapiens
ggggtggcgcccggagctgcacggagagcgtgcccgtcagtgaccgaagaagagaccaagatgaatgcagagcccgagaggaagtttggcgtggtggtggttggtgttggccgagccggctccgtgcggatgagggacttgcggaatccacacccttcctcagcgttcctgaacctgattggcttcgtgtcgagaagggagctcgggagcattgatggagtccagcagatttctttggaggatgctctttccagccaagaggtggaggtcgcctatatctgcagtgagagctccagccatgaggactacatcaggcagttccttaatgctggcaagcacgtccttgtggaataccccatgacactgtcattggcggccgctcaggaactgtgggagctggctgagcagaaaggaaaagtcttgcacgaggagcatgttgaactcttgatggaggaattcgctttcctgaaaaaagaagtggtggggaaagacctgctgaaagggtcgctcctcttcacatctgacccgttggaagaagaccggtttggcttccctgcattcagcggcatctctcgactgacctggctggtctccctctttggggagctttctcttgtgtctgccactttggaagagcgaaaggaagatcagtatatgaaaatgacagtgtgtctggagacagagaagaaaagtccactgtcatggattgaagaaaaaggacctggtctaaaacgaaacagatatttaagcttccatttcaagtctgggtccttggagaatgtgccaaatgtaggagtgaataagaacatatttctgaaagatcaaaatatatttgtccagaaactcttgggccagttctctgagaaggaactggctgctgaaaagaaacgcatcctgcactgcctggggcttgcagaagaaatccagaaatattgctgttcaaggaagtaagaggaggaggtgatgtagcacttccaagatggcaccagcatttggttcttctcaagagttgaccattatctctattcttaaaattaaacatgttggggaaacaaaaaaaaaaaaaaaaa

SEQ ID NO: 3 - protein, 296 aa, Homo sapiens
MNTEPERKFGVVVVGVGRAGSVRMRDLRNPHPSSAFLNLIGFVSRRELGSIDGVQQISLEDALSSQEVEVAYICSESSSHEDYIRQFLNAGKHVLVEYPMTLSLAAAQELWELAEQKGKVLHEEHVELLMEEFAFLKKEVVGKDLLKGSLLFTAGPLEEERFGFPAFSGISRLTWLVSLFGELSLVSATLEERKEDQYMKMTVCLETEKKSPLSWIEEKGPGLKRNRYLSFHFKSGSLENVPNVGVNKNIFLKDQNIFVQKLLGQFSEKELAAEKKRILHCLGLAEEIQKYCCSRK

SEQ ID NO: 4 - protein, 58 aa, Artificial Sequence
  Description of Artificial Sequence  Residues 326-383 Homo sapiens Shaker
GGGGQNGQQAMSLAILRVIRLVRVFRIFKLSRHSKGLQILGKTLQASMRELGLLIFFL

SEQ ID NO: 5 - protein, 58 aa, Artificial Sequence
  Description of Artificial Sequence  Residues 251-308 of Homo sapiens c-Jun
ERIKAERKRMRNRIAASKCRKRKLERIARLEEKVKTLKAQNSELASTANMLREQVAQL

SEQ ID NO: 6 - protein, 58 aa, Artificial Sequence
  Description of Artificial Sequence  Residues 136-193 of Homo sapiens c-Fos
EEEKRRIRRERNKMAAAKCRNRRRELTDTLQAETDQLEDEKSALQTEIANLLKEKEKL

SEQ ID NO: 7 - protein, 58 aa, Artificial Sequence
  Description of Artificial Sequence  Residues 99-156 of Rattus norvegicus BVR
MTLSFAAAQELWELAAQKGRVLHEEHVELLMEEFEFLRREVLGKELLKGSLRFTASPL

SEQ ID NO: 8 - protein, 58 aa, Artificial Sequence
  Description of Artificial Sequence  Residues 377-434 of Homo sapiens c-Myc
LRDQIPELENNEKAPKVVILKKATAYILSVQAEEQKLISEEDLLRKRREQLKHKLEQL

SEQ ID NO: 9 - protein, 58 aa, Artificial Sequence
  Description of Artificial Sequence  Residues 224-281 of Saccharomyces cerevisiae GCN4
SSDPAALKRARNTEAARRSRARKLQRMKQLEDKVEELLSKNYHLENEVARLKKLVGER

SEQ ID NO: 10 - protein, 58 aa, Artificial Sequence
  Description of Artificial Sequence  Residues 282-339 of Homo sapiens CREB
AARKREVRLMKNREAARECRRKKKEYVKCLENRVAVLENQNKTLIEELKALKDLYCHK

SEQ ID NO: 11 - protein, 58 aa, Artificial Sequence
  Description of Artificial Sequence  Residues 63-120 of Saccharomyces cerevisiae YAP
DPETKQKRTAQNRAAQRAFRERKERKMKELEKKVQSLESIQQQNEVEATFLRDQLITL

SEQ ID NO: 12 - DNA, 26 bp, Artificial Sequence
  Description of Artificial Sequence oligonucleotide
ggatccatgaatgcagagcccgagag

SEQ ID NO: 13 - DNA, 26 bp, Artificial Sequence
  Description of Artificial Sequence oligonucleotide
ctcgagagctacatcacctcctcctc

SEQ ID NO: 14 - DNA, 25 bp, Artificial Sequence
  Description of Artificial Sequence oligonucleotide
ggatccatggagcgcccacagctcg

SEQ ID NO: 15 - DNA, 38 bp, Artificial Sequence
  Description of Artificial Sequence oligonucleotide
gctcgagtggcgaaggatcaccatcgcaggagcggtgt

SEQ ID NO: 16 - DNA, 40 bp, Artificial Sequence
  Description of Artificial Sequence oligonucleotide
gaaaaaagaagtggtgggggctgacctgctgaaagggtcg

SEQ ID NO: 17 - DNA, 40 bp, Artificial Sequence
  Description of Artificial Sequence oligonucleotide
cgaccctttcagcaggtcagcccccaccacttcttttttc

SEQ ID NO: 18 - DNA, 39 bp, Artificial Sequence
  Description of Artificial Sequence oligonucleotide
gacctgctgaaagggtcggccctcttcacatctgacccg

SEQ ID NO: 19 - DNA, 39 bp, Artificial Sequence
  Description of Artificial Sequence oligonucleotide
cgggtcagatgtgaagagggccgaccctttcagcaggtc

SEQ ID NO: 20 - DNA, 41 bp, Artificial Sequence
  Description of Artificial Sequence oligonucleotide
cctcttcacatctgacccggctgaagaagaccggtttggct

SEQ ID NO: 21 - DNA, 41 bp, Artificial Sequence
  Description of Artificial Sequence oligonucleotide
agccaaaccggtcttcttcagccgggtcagatgtgaagagg

SEQ ID NO: 22 - DNA, 100 bp, Artificial Sequence
  Description of Artificial Sequence oligonucleotide
tcctcagctgcttttatgctgtgtcatggttgggaggggtgattagcagacaaagggaagacagattttgcgatcctcccctctgttccctctgcctcag

SEQ ID NO: 23 - DNA, 99 bp, Artificial Sequence
  Description of Artificial Sequence oligonucleotide
ctgaggcagagggaacagaggggaggatcgcaaaatctgtcttccctttgtctgctaatcacccctcccaaccatacacagcataaaagcagctgagga

SEQ ID NO: 24 - DNA, 21 bp, Artificial Sequence
  Description of Artificial Sequence oligonucleotide
cagccatgaggactacatcag

SEQ ID NO: 25 - DNA, 21 bp, Artificial Sequence
  Description of Artificial Sequence oligonucleotide
agccagttccttctcagagaa

SEQ ID NO: 26 - DNA, 100 bp, Artificial Sequence
  Description of Artificial Sequence oligonucleotide
tcctcagctgcttttatgctgtgtcatggttgggaggggtgattagcagacaaagggaagacagattttgctgagtcaccctctgttccctctgcctcag

SEQ ID NO: 27 - DNA, 100 bp, Artificial Sequence
  Description of Artificial Sequence oligonucleotide
ctgaggcagagggaacagagggtgactcagcaaaatctgtcttccctttgtctgctaatcacccctcccaaccatgacacagcataaaagcagctgagga

SEQ ID NO: 28 - DNA, 100 bp, Artificial Sequence
  Description of Artificial Sequence oligonucleotide
tcctcagctgcttttatgcgatcctctggttgggaggggtgattagcagacaaagggaagacagattttgcgatcctcccctctgttccctctgcctcag

SEQ ID NO: 29 - DNA, 100 bp, Artificial Sequence
  Description of Artificial Sequence oligonucleotide
ctgaggcagagggaacagaggggaggatcgcaaaatctgtcttccctttgtctgctaatcacccctcccaaccagaggatcgcataaaagcagctgagga

SEQ ID NO: 30 - DNA, 56 bp, Artificial Sequence
  Description of Artificial Sequence oligonucleotide
cactgagagaaactattacacaagccacattagcatgactcattgtttctgatcag

SEQ ID NO: 31 - DNA, 56 bp, Artificial Sequence
  Description of Artificial Sequence oligonucleotide
ctgatcagaaacaatgagtcatgctaatgtggcttgtgtaatagtttctctcagtg

SEQ ID NO: 32 - DNA, 56 bp, Artificial Sequence
  Description of Artificial Sequence oligonucleotide
cactgagagaaactattacacaagccacattagcagatcctcttgtttctgatcag

SEQ ID NO: 33 - DNA, 56 bp, Artificial Sequence
  Description of Artificial Sequence oligonucleotide
ctgatcagaaacaagaggatctgctaatgtggcttgtgtaatagtttctctcagtg

SEQ ID NO: 34 - DNA, 36 bp, Artificial Sequence
  Description of Artificial Sequence  primer
gcggcggctggtgttgcgcgcgccggctccgtgcgg

SEQ ID NO: 35 - DNA, 36 bp, Artificial Sequence
  Description of Artificial Sequence  primer
ccgcacggagccggcgcgcgcaacaccagccgccgc

SEQ ID NO: 36 - DNA, 44 bp, Artificial Sequence
  Description of Artificial Sequence  primer
cctgctgaaaggggcggccgccgccgcagctgacccgttggaag

SEQ ID NO: 37 - DNA, 44 bp, Artificial Sequence
  Description of Artificial Sequence  primer
cttccaacgggtcagctgcggcggcggccgcccctttcagcagg